(19) United States
(12) Patent Application Publication
(10) Pub. No.: US 2024/0318161 A1
(43) Pub. Date: Sep. 26, 2024

(54) COMPOSITIONS AND METHODS FOR NUCLEIC ACID EXTRACTION AND LIBRARY PREPARATION

(71) Applicant: ILLUMINA, INC., San Diego, CA (US)

(72) Inventors: Vicki Thomson, Upper Cambourne (GB); Maria Vitoriano, Baldock (GB); Sébastien Ricoult, Sawston (GB); Johan Basuki, Singapore (SG)

(21) Appl. No.: 18/613,690

(22) Filed: Mar. 22, 2024

Related U.S. Application Data
(60) Provisional application No. 63/492,081, filed on Mar. 24, 2023.

(51) Int. Cl. C12N 15/10; C40B 50/00

(52) U.S. Cl. CPC C12N 15/1003; C40B 50/00

(57) ABSTRACT

The present disclosure provides systems and methods for preparing a DNA library from a sample comprising shelf-stable lyophilized microspheres comprising DNA library preparation reagents providing a streamlined workflow for DNA library preparation. The present disclosure also provides a smart consumable container for collecting and transporting the sample.

### CROSS REFERENCE TO RELATED APPLICATIONS

The present application claims the priority benefit of U.S. Provisional Patent Application No. 63/492,081, filed Mar. 24, 2023, incorporated by reference herein in its entirety.

### FIELD

The present disclosure relates generally to compositions comprising nucleic acid library preparation reagents, systems, and methods of use thereof in the preparation of nucleic acid libraries. In particular, the present disclosure provides materials adapted to deliver and release lyophilized reagents to a sample for sequential nucleic acid extraction and library preparation in a streamlined process for a variety of downstream applications including, e.g., next generation DNA sequencing.

### BACKGROUND

With the advent of massively parallel short-read sequencing technologies, also known as Next Generation Sequencing (NGS) (Bentley et al., 2008, Nature. 456(7218): 53-59), the cost of sequencing DNA has reduced by orders of magnitude. Moreover, the very high throughput data acquisition with NGS has allowed for rapid sequencing of complete genomes with unprecedented ease, providing access to increasing amounts of genomic, transcriptomic, and epigenetic data across all fields of biology.

NGS-based projects can be roughly divided into the following process elements that should be tailored and optimized to the target nucleic acid (RNA or DNA) and sequencing system selected; sample pre-processing for nucleic acid extraction (NAE), library preparation, sequencing itself/data acquisition and bioinformatics. Both NAE and library preparation remain lengthy, multi-step, low-throughput processes. NAE can be roughly divided into four steps, which can be modulated depending on the sample and downstream applications: (i) cell disruption; (ii) removal of membrane lipids, proteins, and other nucleic acids, (iii) nucleic acid purification/binding from bulk; and (iv) nucleic acid concentration. Library preparation is an essential process that comprises several aspects that affect the efficiency of NGS. It often involves the following main steps: fragmentation of the input DNA, end-repair and A-tailing of the DNA fragments, ligation of indexed sequencing adapters and optional amplification of the ligated products. In addition, one or more cleanup steps may be useful in between steps to purify the DNA reaction products of reagents from the previous reaction. Alternate methods include fragmentation-based library prep here (e.g., tagmentation) in which the adapters can be added during the fragmentation step, and no A-tailing is necessary. Reliable and standardized implementation and quality control measures for all stages of the process are crucial.

Challenges are encountered at each of the aforementioned workflow steps and benefits and advantages may be realized by tackling these challenges to enable high quality sequencing results. For instance, the extraction of a sufficient amount of good quality DNA, which is free from inhibitors of enzymatic reactions that occur downstream in the NGS workflow, can vary in complexity depending on the sample type and its storage conditions. During library preparation, three major challenges can be observed: complexity of protocols, imprecise pipetting, contamination and cost. The bead-based purification steps in particular, which entail the handling of magnets and magnetic particles by the user, are error-prone and can result in failure of the library preparation (Meyer and Kircher., 2010 Cold Spring Harb Protoc. 2010 (6):pdb.prot5448). Sample contamination is an inherent problem, as libraries are usually prepared in parallel (Kotrova et al., 2017. Mol Diagn Ther. 21(5):481-492; Salter et al., 2014. BMC Biol.12:87). Major sources of contamination are pre-amplifications required for low starting concentration of nucleic acids (Kotrova et al., 2017. Mol Diagn Ther. 21(5):481-492). Multiple liquid-handling steps also increase the risk of sample cross-contamination.

Thus this standard NGS workflow is both complex and expensive requiring expensive laboratory equipment and reagents, trained personnel, and also involves many liquid-handling steps. So even as the cost of data acquisition (sequencing) continues to decrease, for many large-scale genomic experiments, sample acquisition, sample storage and requisite cold chain, sample pre-processing and library preparation for sequencing lag behind in their progress creating a time, cost, and labor bottleneck. This represents a severe constraint in resource-limited settings, large or geographically dispersed healthcare networks, and military or government-funded public health laboratories. As a result, large swathes of diverse, multi-ethnic populations continue to be underrepresented in in health-related genetic research, potentially exacerbating existing disease and healthcare disparities (Bentley et al., 2019, Ethn Dis., 29(Suppl 1):179-186; Wojcik et al., 2019, Nature 570: 514-518).

To increase the diversity of participants and investigators in genomic research and harness the full potential of current sequencing technology, there is need for lowering reagent, sample storage and shipping costs as well as simplified sample pre-processing and library preparation workflows to reduce the number of liquid handling steps and required hands-on time. Streamlining sample preprocessing and library preparation could make NGS affordable for any laboratory and health system around the world.

### SUMMARY

Described herein are methods that integrate DNA extraction, fragmentation and indexing to reduce the time and complexity of template preparation, enabling higher throughput and process cost reductions compared to conventional methods.

Provided herein is a system for collecting and preparing nucleic acids from a biological sample for DNA library amplification comprising a container with an opening configured to receive a biological sample, wherein the container comprises a workflow reagent release system, comprising: a. one or more lyophilized microspheres comprising lysis buffer and a proteinase; and, b. a plurality of first particles comprising i) a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; ii) a second outer shell which encapsulates a second inner core comprising one or more lyophilized microspheres comprising one or more reagents for tagmentation of DNA, wherein the second outer shell releases the second inner core in response to a second release trigger mechanism.

Also contemplated is a system for collecting and preparing nucleic acids from a biological sample for DNA library amplification comprising a container with an opening configured to receive a biological sample, wherein the container comprises a workflow reagent release system, comprising: a. one or more lyophilized microspheres comprising lysis buffer and a thermolabile proteinase; and b. a plurality of first particles comprising i) a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; ii) a second outer shell which encapsulates a second inner core comprising one or more lyophilized microspheres comprising one or more reagents for tagmentation of DNA, wherein the second outer shell releases the second inner core in response to a second release trigger mechanism.

Provided herein is a system for collecting and preparing nucleic acids from a biological sample for DNA library amplification comprising a container with an opening configured to receive a biological sample, wherein the container comprises a workflow reagent release system, comprising: a. a plurality of lyophilized microspheres comprising lysis buffer and a proteinase; and, b. a plurality of first particles comprising i) a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; ii) a second outer shell which encapsulates a second inner core comprising one or more lyophilized microspheres comprising one or more reagents for tagmentation of DNA, wherein the second outer shell releases the second inner core in response to a second release trigger mechanism.

Also contemplated is a system for collecting and preparing nucleic acids from a biological sample for DNA library amplification comprising a container with an opening configured to receive a biological sample, wherein the container comprises a workflow reagent release system, comprising: a. a plurality of lyophilized microspheres comprising lysis buffer and a thermolabile proteinase; and b. a plurality of first particles comprising i) a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; ii) a second outer shell which encapsulates a second inner core comprising one or more lyophilized microspheres comprising one or more reagents for tagmentation of DNA, wherein the second outer shell releases the second inner core in response to a second release trigger mechanism.

Also provided herein is a system for collecting and preparing nucleic acids from a biological sample for DNA library amplification comprising a container with an opening configured to receive a biological sample, wherein the container comprises a workflow reagent release system, comprising: a. a lyophilized cake comprising lysis buffer and a proteinase; and, b. a plurality of first particles comprising i) a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; ii) a second outer shell which encapsulates a second inner core comprising one or more lyophilized microspheres comprising one or more reagents for tagmentation of DNA, wherein the second outer shell releases the second inner core in response to a second release trigger mechanism.

Also contemplated is a system for collecting and preparing nucleic acids from a biological sample for DNA library amplification comprising a container with an opening configured to receive a biological sample, wherein the container comprises a workflow reagent release system, comprising: a. a lyophilized cake comprising lysis buffer and a thermolabile proteinase; and b. a plurality of first particles comprising i) a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; ii) a second outer shell which encapsulates a second inner core comprising one or more lyophilized microspheres comprising one or more reagents for tagmentation of DNA, wherein the second outer shell releases the second inner core in response to a second release trigger mechanism.

In various implementations, the first particle optionally comprises a third outer shell which encapsulates a third inner core, wherein the third inner core comprises workflow reagents for extension-ligation and PCR, wherein the third outer shell releases the third inner core in response to a third release trigger mechanism.

In various implementations, the system further comprising a plurality of second particles comprising a third outer shell which encapsulates a third inner core, wherein the third inner core comprises workflow reagents for extension-ligation and PCR, wherein the third outer shell releases the third inner core in response to a third release trigger mechanism.

In various implementations, the first and second outer shells are only sensitive to the first or second release trigger mechanisms respectively.

In various implementations, the first trigger release mechanism is the biological sample dissolving the lyophilized microspheres, thereby forming a lysis solution.

In various implementations, the first lyophilized microspheres comprise one or more reagents for lysing cells contained in the biological sample. In various implementations, the one or more first lyophilized microspheres comprise one or more reagents for lysing cells contained in the biological sample. In various implementations, the one or more reagents for lysing cells is selected from the group consisting of a phosphate buffer solution, a salt, a detergent, an alcohol, a protease, a lysis buffer, a lyoprotectant, or a combination thereof.

In various implementations, the lyophilized cake comprises one or more reagents for lysing cells contained in the biological sample. In various implementations, the one or more reagents for lysing cells is selected from the group consisting of a phosphate buffer solution, a salt, a detergent, an alcohol, a protease, a lysis buffer, a lyoprotectant, or a combination thereof. In various implementations, the proteinase is a broad-spectrum serine proteinase. In various implementations, the broad-spectrum serine proteinase is Proteinase K, optionally a thermolabile Proteinase K.

In various implementations, the detergent is sodium dodecyl sulfate (SDS).

In various implementations, the lyophilized cake comprises sodium dodecyl sulfate (SDS), EDTA, Tween and a proteinase.

In various implementations, the first, second or third release trigger is a temperature-controlled release mechanism, a pH-controlled release mechanism, a time-controlled release mechanism, a position-controlled release mechanism, or any combination thereof. In some implementations, the second trigger release mechanism comprises a temperature-controlled trigger release or a time-controlled trigger release.

In various implementations, the second inner core comprises one or more lyophilized microspheres comprising one or more tagmentation reagents. In various implementations, the one or more tagmentation reagents are selected from the group consisting of a Tn5 transposase enzyme, one or more transposons, linker sequences, Tn5 2×Tagmentation Buffer, Mg2+, an SDS chelating agent, primers with transposome, a lyoprotectant and optionally, a Proteinase K inhibitor. In various implementations, the SDS chelating agent is a cyclodextrin (CD) selected from the group consisting of α-CD, β-CD, and γ-CD.

In various implementations, the first, second and/or if present the third outer shells comprises of one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, Eudragit®, cellulose acetate, ethyl cellulose, UCST and LCST polymers, or any combination thereof.

In various implementations, the lyoprotectant is selected from the group consisting of mannitol, sorbitol, inositol, sucrose, glucose, mannose and trehalose.

In various implementations, the biological sample is blood.

In various implementations, the nucleic acid is DNA. In various implementations, the DNA is genomic DNA (gDNA).

The disclosure provides a method for preparing a nucleic acid library from a biological sample comprising: A. collecting a biological sample from a subject and placing the sample into a container with an opening configured to receive a biological sample, wherein the container comprises a workflow reagent release system; wherein the workflow reagent release system comprises a first lyophilized microspheres comprising lysis buffer and a proteinase; and b. first particles comprising i) a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; ii) a second outer shell which encapsulates a second inner core comprising one or more lyophilized microspheres comprising one or more reagents for tagmentation of DNA, wherein the second outer shell releases the second inner core in response to a second release trigger mechanism; wherein the biological sample interacts with the lyophilized lysis buffer in the container resulting in release of nucleic acid from cells in the biological sample, and allowing the lysis buffer to react for a period of time sufficient to carry out lysis of cells in the biological sample; B. inactivating the lysis reaction (A) after the period of time by activating the first release trigger mechanism to release the proteinase inhibitor and detergent chelator from the first inner core, and allowing the inactivation reaction to proceed for a period of time sufficient to inactive the proteinase and to chelate the detergent; and C. stopping the inactivation reaction (B) by activating the second release trigger mechanism to release the reagents for tagmentation of DNA from the second inner core, and allowing the tagmentation reaction to proceed for a period of time sufficient to tag the nucleic acid from the biological sample.

In various implementations, the method further comprises isolating the nucleic acid from (C) and generating a nucleic acid library using a library preparation kit.

The disclosure provides a method for preparing a nucleic acid library from a biological sample comprising: A. collecting a biological sample from a subject and placing the sample into a container with an opening configured to receive a biological sample, wherein the container comprises a workflow reagent release system; wherein the workflow reagent release system comprises a. a lyophilized cake comprising lysis buffer and a proteinase; and b. first particles comprising i) a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; ii) a second outer shell which encapsulates a second inner core comprising one or more lyophilized microspheres comprising one or more reagents for tagmentation of DNA, wherein the second outer shell releases the second inner core in response to a second release trigger mechanism; wherein the biological sample interacts with the lyophilized lysis buffer in the container resulting in release of nucleic acid from cells in the biological sample, and allowing the lysis buffer to react for a period of time sufficient to carry out lysis of cells in the biological sample; B. inactivating the lysis reaction (A) after the period of time by activating the first release trigger mechanism to release the proteinase inhibitor and detergent chelator from the first inner core, and allowing the inactivation reaction to proceed for a period of time sufficient to inactive the proteinase and to chelate the detergent; and C. stopping the inactivation reaction (B) by activating the second release trigger mechanism to release the reagents for tagmentation of DNA from the second inner core, and allowing the tagmentation reaction to proceed for a period of time sufficient to tag the nucleic acid from the biological sample.

In various implementations, the method further comprises isolating the nucleic acid from (C) and generating a nucleic acid library using a library preparation kit.

In various implementations, the first and second outer shells are only sensitive to the first or second release trigger mechanisms respectively.

In various implementations, the first trigger mechanism is initiated when the biological sample dissolves the lyophilized microspheres comprising lysis buffer.

In various implementations, the first lyophilized microspheres comprise one or more reagents for lysing cells in a biological sample. In various implementations, the one or more first lyophilized microspheres comprise one or more reagents for lysing cells in a biological sample. In various implementations, the one or more reagents for lysing cells is selected from the group consisting of a phosphate buffer solution, a salt, a detergent, an alcohol, a protease, a lysis buffer, a lyoprotectant, or a combination thereof.

In various implementations, the proteinase is a broad-spectrum serine proteinase. In various implementations, the broad-spectrum serine proteinase is Proteinase K, optionally a thermolabile Proteinase K.

In various implementations, the detergent is sodium dodecyl sulfate (SDS).

In various implementations, the first, second or third release trigger if present is a temperature-controlled release mechanism, a pH-controlled release mechanism, a time-controlled release mechanism, a position-controlled release mechanism, or any combination thereof. In various implementations, the second trigger release mechanism comprises a temperature-controlled trigger release or a time-controlled trigger release.

In various implementations, the second inner core comprises one or more lyophilized microspheres comprising one or more tagmentation reagents. In various implementations, the one or more tagmentation reagents are selected from the group consisting of a Tn5 transposase enzyme, one or more transposons, linker sequences, Tn5 2× Tagmentation Buffer, Mg2+, an SDS chelating agent, primers with transposome, a lyoprotectant and optionally, a proteinase inhibitor.

In various implementations, the second inner core comprises one or more lyophilized microspheres comprising one or more tagmentation reagents. In various implementations, the one or more tagmentation reagents are selected from the group consisting of a Tn5 transposase enzyme, one or more transposons, linker sequences, Tn5 2× Tagmentation Buffer, Mg2+, an SDS chelating agent, primers with transposome, a lyoprotectant and optionally, a Proteinase K inhibitor.

In various implementations, the one or more tagmentation reagents are selected from the group consisting of a Tn5 transposase enzyme, one or more transposons, linker sequences, Tn5 2× Tagmentation Buffer, Mg2+, an SDS chelating agent, primers with transposome, and a lyoprotectant. In various implementations, the thermolabile proteinase is inhibited by the temperature of the tagmentation reaction. In various implementations, the temperature is between 50° C. and 80° C., e.g., for 10 minutes.

In various implementations, the SDS chelating agent is a cyclodextrin (CD) selected from the group consisting of α-CD, β-CD, and γ-CD.

In various implementations, the tagmentation reaction occurs between about 25° C. and about 55° C. In various implementations, the tagmentation reaction occurs between about 30° C. and about 50° C., or between about 37° C. and about 45° C., or between about 40° C. and about 50° C. In various implementations, the tagmentation reaction occurs at about 25° C., at about 30° C., at about 35° C., about 36° C., at about 37° C., at about 38° C., at about 39° C., at about 40° C., at about 41° C., at about 42° C., at about 43° C., at about 44° C., at about 45° C., at about 46° C., at about 47° C., at about 48° C., at about 49° C., or at about 50° C.

In various implementations, the first, second and/or third outer shells if present comprises of one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, Eudragit®, cellulose acetate, ethyl cellulose, UCST and LCST polymers or any combination thereof.

In various implementations, the lyoprotectant is selected from the group consisting of mannitol, sorbitol, inositol, sucrose, glucose, mannose and trehalose.

In various implementations, the biological sample is blood.

In various implementations, the nucleic acid is DNA. In various implementations, the DNA is genomic DNA (gDNA).

In various implementations, the method comprises (a) contacting the sample with a first lyophilized microsphere comprising a lysis reagent that generates a cell lysate, wherein the lysis reagent has one or more proteases, and wherein the cell lysate contains a target nucleic acid. In various implementations, the release of the tagmentation reagents applies at least one transposase and at least one transposon end composition containing a transferred strand under conditions where the target nucleic acid and the transposon end composition undergo a transposition reaction to generate a mixture, wherein, the target nucleic acid is fragmented to generate a plurality of target nucleic acid fragments, and the transferred strand of the transposon end composition is joined to 5′ ends of each of a plurality of the target nucleic acid fragments to generate a plurality of 5′ tagged target nucleic acid fragments.

In various implementations, the method comprises (a) contacting the sample with a lyophilized cake comprising a lysis reagent that generates a cell lysate, wherein the lysis reagent has one or more proteases, and wherein the cell lysate contains a target nucleic acid. In various implementations, the release of the tagmentation reagents applies at least one transposase and at least one transposon end composition containing a transferred strand under conditions where the target nucleic acid and the transposon end composition undergo a transposition reaction to generate a mixture, wherein, the target nucleic acid is fragmented to generate a plurality of target nucleic acid fragments, and the transferred strand of the transposon end composition is joined to 5′ ends of each of a plurality of the target nucleic acid fragments to generate a plurality of 5′ tagged target nucleic acid fragments.

In various implementations, the target nucleic acid is a double-stranded DNA. In various embodiments, the target nucleic acid remains the double-stranded DNA prior to applying a transposase and a transposon end composition.

Also provided herein is a container for collecting a biological sample, the container comprising a workflow reagent release system as described herein, wherein the container comprises an indicator that changes upon completion of the workflow reagent release system inside the container.

In various implementations, the container comprises a radio-frequency identification (RFID) tag. In various implementations, the RFID tag is embedded on the container, optionally wherein the RFID has the capacity to store at least 8 kilobytes of information,

In various implementations, the container comprises an opening for receiving a biological sample comprising nucleic acids.

In various implementations, the container comprises a heating element and a temperature sensor coupled to said container, and wherein the container's RFID tag stores a temperature history,

In various implementations, the container is tamper proof.

In various implementations, the container is made from polypropylene or cyclic olefin copolymer. In various embodiments, the container is a PCR tube, vial, microtube, flow cell, multiwell plate, glass tube, cartridge or microfluidic chip.

Further contemplated by the disclosure is a method of transporting a sample for preparation of a nucleic acid library, comprising: inserting the sample into a container configured to receive a biological sample, wherein the container comprises a system as described herein; sealing the container such that the system begins the process of sample lysis and nucleic acid library preparation; shipping the sealed container to a nucleic acid sequencing laboratory such that upon arrival at the sequencing laboratory, the system has completed lysis of the sample, tagmentation of nucleic acid in the sample, extension-ligation and PCR of nucleic acid in the sample.

In various implementations, the container is stored between about 4° C. and 30° C. at the inserting step, the sealing step and or the shipping step. In various implementations, the container is stored between about 4° C. and 8° C., between about 4° C. and 25° C., or between about 20° C. and 30° C. at the inserting step, the sealing step and or the shipping step.

In various implementations, the method of transporting further comprises removing the container from the shipment and isolating the nucleic acid from the sample in the container. In various implementations, the container comprises an RFID tag. In various implementations, the container further comprises an indicator showing the library preparation is complete.

In various implementations, the biological sample is blood.

In various implementations, the nucleic acid is DNA. In various implementations, the DNA is genomic DNA (gDNA).

Also provided is a composition comprising: a. a plurality of first lyophilized microspheres comprising lysis buffer and a proteinase; and b. a plurality of first particles comprising i) a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; ii) a second outer shell which encapsulates a second inner core comprising one or more lyophilized microspheres comprising one or more reagents for tagmentation of DNA, wherein the second outer shell releases the second inner core in response to a second release trigger mechanism.

Further contemplated is a composition comprising: a. a plurality of first lyophilized microspheres comprising lysis buffer and a thermolabile proteinase; and b. a plurality of first particles comprising i) a first outer shell which encapsulates a first inner core, wherein the first inner core comprises one or more lyophilized microspheres comprising an inhibitor of the proteinase and a detergent chelator; ii) a second outer shell which encapsulates a second inner core comprising one or more lyophilized microspheres comprising one or more reagents for tagmentation of DNA.

Also provided is a composition comprising: a. a lyophilized cake comprising lysis buffer and a proteinase; and b. a plurality of first particles comprising i) a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; ii) a second outer shell which encapsulates a second inner core comprising one or more lyophilized microspheres comprising one or more reagents for tagmentation of DNA, wherein the second outer shell releases the second inner core in response to a second release trigger mechanism.

Further contemplated is a composition comprising: a. a lyophilized cake comprising lysis buffer and a thermolabile proteinase; and b. a plurality of first particles comprising i) a first outer shell which encapsulates a first inner core, wherein the first inner core comprises one or more lyophilized microspheres comprising an inhibitor of the proteinase and a detergent chelator; ii) a second outer shell which encapsulates a second inner core comprising one or more lyophilized microspheres comprising one or more reagents for tagmentation of DNA.

In various implementations, the first particle optionally comprises a third outer shell which encapsulates a third inner core, wherein the third inner core comprises workflow reagents for extension-ligation and PCR, wherein the third outer shell releases the third inner core in response to a third release trigger mechanism.

In various implementations, the composition further comprises a plurality of second particles comprising a third outer shell which encapsulates a third inner core, wherein the third inner core comprises workflow reagents for extension-ligation and PCR, wherein the third outer shell releases the third inner core in response to a third release trigger mechanism.

In various implementations, the first outer shell releases the first inner core in response to a first release trigger mechanism, wherein the second outer shell releases the second inner core in response to a second release trigger mechanism, and wherein if there is a third release trigger mechanism wherein the third outer shell releases the third inner core in response to a third release trigger mechanism. In various implementations, the first trigger release mechanism is triggered by a biological sample dissolving the lyophilized microspheres, thereby forming a lysis solution.

In various implementations, the first lyophilized microspheres comprise one or more reagents for lysing cells. In various implementations, the one or more reagents for lysing cells is selected from the group consisting of a phosphate buffer solution, a salt, a detergent, an alcohol, a protease, a lysis buffer, a lyoprotectant, or a combination thereof.

In various implementations, the lyophilized cake comprises one or more reagents for lysing cells. In various implementations, the one or more reagents for lysing cells is selected from the group consisting of a phosphate buffer solution, a salt, a detergent, a protease, a lysis buffer, a lyoprotectant, or a combination thereof.

In various implementations, the proteinase is a broad-spectrum serine proteinase. In various implementations, the broad-spectrum serine proteinase is Proteinase K, optionally a thermolabile Proteinase K.

In various implementations, the detergent is sodium dodecyl sulfate (SDS).

In various implementations, the lyophilized cake comprises sodium dodecyl sulfate (SDS), EDTA, Tween and a proteinase.

In various implementations, the first, second or third release trigger is a temperature-controlled release mechanism, a pH-controlled release mechanism, a time-controlled release mechanism, a position-controlled release mechanism, or any combination thereof.

In various implementations, the second trigger release mechanism comprises a temperature-controlled trigger release or a time-controlled trigger release.

In various implementations, the second inner core comprises lyophilized microspheres comprising one or more tagmentation reagents. In various implementations, the one or more tagmentation reagents are selected from the group consisting of a Tn5 transposase enzyme, one or more transposons, linker sequences, Tn5 2× Tagmentation Buffer, Mg2+, an SDS chelating agent, primers with transposome, a lyoprotectant and optionally, a Proteinase K inhibitor. In various implementations, the SDS chelating agent is a cyclodextrin (CD) selected from the group consisting of α-CD, β-CD, and γ-CD.

In various implementations, the first, second and/or third outer shells if present comprises of one or more of polyvinyl alcohol, polyvinylpyrrolidone (PVP), carrageenan, gelatin, hydroxypropyl methylcellulose (HPMC), pullulan, starch film, benzoxaborole-poly(vinyl alcohol) (benzoxaborole-PVA), pectin, Eudragit®, cellulose acetate, ethyl cellulose, UCST and LCST polymers, or any combination thereof.

In various implementations, the lyoprotectant is selected from the group consisting of mannitol, sorbitol, inositol, sucrose, glucose, mannose and trehalose.

It is understood that each feature or embodiment, or combination, described herein is a non-limiting, illustrative example of any of the aspects of the invention and, as such, is meant to be combinable with any other feature or implementation, or combination, described herein. For example, where features are described with language such as “one implementation”, “various implementations”, “some implementations”, “certain implementations”, “further implementation”, “specific exemplary implementations”, and/or “another implementation”, each of these types of implementations is a non-limiting example of a feature that is intended to be combined with any other feature, or combination of features, described herein without having to list every possible combination.

Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

The drawings illustrate one or more disclosed embodiments and serve to explain the principles of the disclosed implementation. It is to be understood, however, that the drawings are designed for purposes of illustration only, and not as a definition of the limits of the invention.

FIG. 1 (bottom panel) schematically illustrates workflows for sample lysis, DNA tagmentation in accordance with one implementation of the disclosure. FIG. 1 (upper panel) schematically illustrates the previous workflow for tagmentation and indexing, that have now been integrated with sample lysis into one simplified workflow.

FIG. 2 is a diagram depicting a simplified representation of the steps and conditions for a one-pot workflow in accordance with one implementation of the present disclosure.

FIG. 3 is a schematic perspective diagram illustrating one implementation of a modular system that can be used to practice aspects of the present disclosure.

FIG. 4 is a diagrammatic illustration of an implementation of a radio frequency identification (“RFID”) tagged container for collecting and transporting a biological sample, wherein the container comprises lyophilized microspheres and core/shell particles that comprise lyophilized microspheres of reagents for passive processing of the sample during transportation

FIG. 5 is a diagrammatic illustration of an implementation of a RFID tagged container comprising lyophilized microspheres and core/shell reagents of the present disclosure for lysis, inactivation and tagmentation of the sample and passively processing a biological sample in accordance with one implementation of the present disclosure.

FIG. 6 is a diagrammatic illustration of an implementation of a RFID tagged container comprising the core/shell reagents of the present disclosure for collecting, transporting and passively processing a biological sample in accordance with one implementation of the present disclosure for one-pot library preparation.

FIGS. 7A-7D provide characterization of lyophilizates from lysis reagent formulations after freeze-drying. The vial depicted in FIG. 7A represents drying below collapse temperature, whereas the vial depicted in FIG. 7B shows a lyophilizate freeze-dried well below the glass transition temperature (Tg′). FIG. 7C is a scatter plot of Tg′ vs the concentration of total solutes, showing that the presence or absence of Proteinase K from the lyophilization mixture does not affect the Tg′ significantly. FIG. 7D shows bright-field microscopy images, at 1× (upper image) and 10× (lower image) magnifications, of exemplary cores pre-coating according to one or more implementations of the present disclosure.

FIGS. 8A-8C depict an exemplary study on the compatibility of the Proteinase K with the polymeric shell. FIG. 8A is a schematic illustration of the procedural steps used for said study, wherein a standard extraction proteinase K/SDS method is used to extract DNA from a blood sample spiked with 5 μL or 20 μL of the coating, DNA purification using Solid Phase Reversible Immobilization (SPRI) Magnetic Beads and DNA quantification. FIG. 8B is a bar graph showing quantification of DNA extracted under a control condition without (Lane 1, left to right) or with Proteinase K (lane 2), or under test conditions, wherein the blood samples had been spiked with 5 μL or 20 μL coating material prior to DNA extraction. The results indicate minimal interference of the coating with the lysis at the higher coating concentration (Lane 4). FIG. 8C lists the experimental conditions in FIG. 8B.

FIG. 9A is a comprehensive table comparing inactivation ability with heat or inhibitor inactivation, presence/absence of glycerol, and DNA yield at 37° C. for each PK candidate evaluated herein. FIG. 9B is an exemplary workflow for performance evaluation of the said proteases, comprising the steps of i) contacting a 25 μL blood sample with a lysis buffer comprising each of the candidate PKs, ii) cell lysis and DNA release at 37° C. for 15 min, iii) PK inactivation, iv) purification of extracted DNA using Solid Phase Reversible Immobilization (SPRI) magnetic beads, and v) measuring the yield of total DNA using a Qubit fluorometer. FIG. 9C shows the effect of the different inhibitors on the standard Illumina DNA prep, which is tagmentation-based.

FIG. 10 shows a boxplot summarizing the distribution of sequencing run quality metrics from the five DNA extractions prepared using the Proteinase Ks. Each subpanel illustrates the results for one quality metric, which listed from the bottom are: (a) insert size (b) Q30 bases % (c) Mapped reads % (d) Coverage (e) clusters passing filter (% PF). The x-axis displays the Proteinase K used for the DNA extraction. Each box plot displays the range and distribution of a quality metric computed for a sequencing run. The top and bottom of the box show the interquartile range and the midline shows the median.

FIGS. 11A and 11B show a graphical comparison of the DNA yields (FIG. 12A) and sequencing run quality metrics of DNA libraries processed using liquid reagents vs the lyophilized reagents according to one or more implementations of the present disclosure (FIG. 11B). FIG. 11B (I) represents control libraries in which purified DNA extracted using standard methods (e.g. Qiagen kit) was used with and without trehalose spiked in to understand the effect of this reagent in the library workflow, FIG. 11B (II) represents samples processed with liquid and lyophilized reagents during the lysis step, wherein the Proteinase K was heat inactivated, FIG. 11B (III) represents libraries processed with liquid and lyophilized reagents during the lysis step, wherein the Proteinase K was irreversibly inhibited using tetrapeptidyl Chloromethyl Ketone (TCK), which targets Proteinase K's active site.

FIGS. 12A-12B depict an exemplary study on the effect of dimethyl sulfoxide (DMSO) on lyophilization (glass transition temperature (Tg′)) and sequencing metrics. FIG. 12A is a scatter plot showing the effect of increasing concentrations of DMSO on the Tg′ of solutions having 20% or 30% solute content. DMSO lowered the Tg′ especially in the solution with the lower solute content. A DMSO concentration of 1% is the most suitable for the buffer formulation since it yields a Tg′ values of above −40° C. FIG. 12B shows boxplots summarizing the distribution of sequencing run quality metrics from a control library prepared with the TL NEB Proteinase K and three test libraries prepared using the Sigma Proteinase K and different concentrations of the TCK prepared using different concentrations of DMSO.

FIG. 13 provides bar graphs showing comparable sequencing QC metrics from libraries prepared with the TCK inhibitor and 0.7%, 0.25%, and 0.08% final concentrations of DMSO.

FIGS. 14A and 14B provides graphs showing sequencing QC metrics from libraries prepared using liquid reagents with or without cyclodextrin in the tagmentation buffer. FIG. 14A compares the performance between liquid and lyophilized tagmentation buffer to show it can be lyophilized and FIG. 14B (liquid only) compares addition of CD+ tagmentation buffer and subsequential addition of TCK versus the addition of the three reagents as a single mix. The results show that the three reagents can potentially be lyophilized together.

FIG. 15 provides a sample method for DNA library preparation using the compositions or systems described herein.

FIG. 16 illustrates one method for transporting a sample in a container or smart consumable as described herein to a facility for sequencing facility.

FIG. 17 shows the effect of lysis buffer on killing of virus from blood samples.

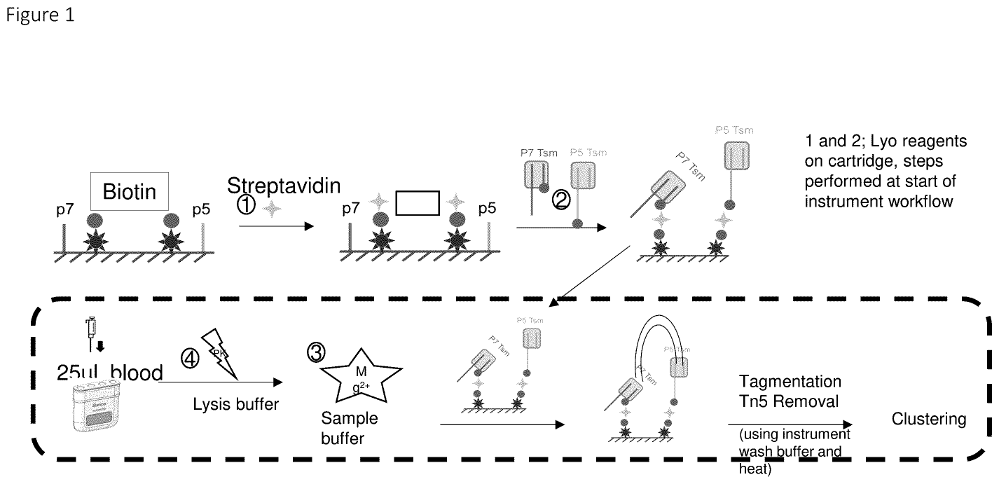

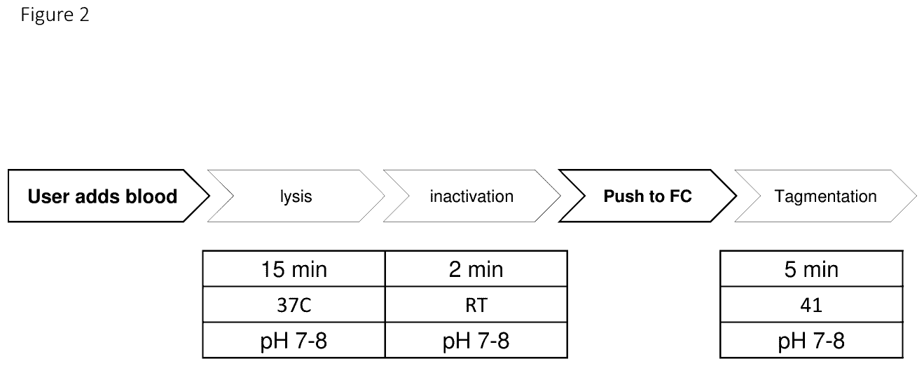

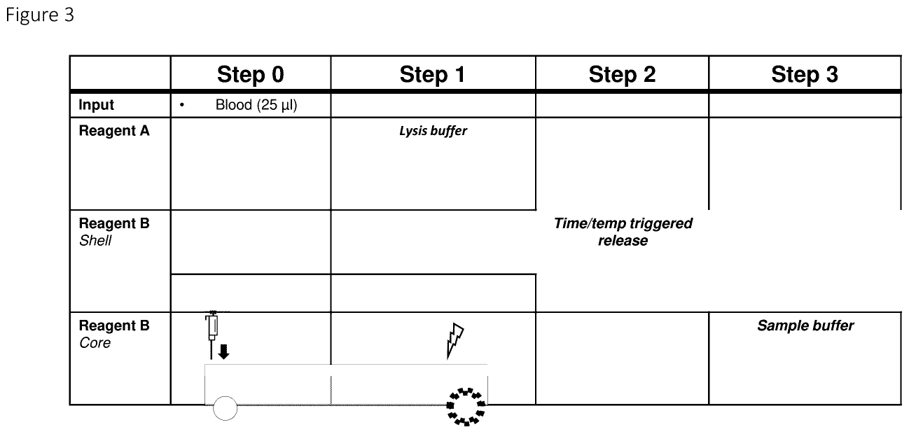

### DETAILED DESCRIPTION

As current technologies allow for sequencing millions or billions of DNA fragments in parallel at relatively low costs, the scope of data generation is often limited by difficulties in sample preparation rather than sequencing capacity. In spite of recent advances, it may be difficult, if at all possible, for nucleic acids to be efficiently sequenced in situ, thus requiring the extraction of nucleic acids from the material under study and their subsequent conversion into DNA libraries. Typically, methods for nucleic acid extraction and preparation of template libraries for next generation sequencing include multiple steps and transfers of reaction volumes between vessels. This makes NGS sample preparation complicated, inefficient and increases the risk of sample cross contamination since samples are prepared in parallel. Furthermore, losses of molecules occur during both steps of sample preparation and impose challenges on work with low nucleic acid input.

The present disclosure addresses the aforementioned shortcomings by providing particles, e.g., comprising a core-shell composite, engineered to deliver and release lyophilized compositions into biological samples for passive nucleic acid extraction and library preparation in a single reaction vessel, i.e., “one-pot format”, or minimal containers or vessels, for a variety of applications including, e.g., next generation DNA sequencing. Provided herein are compositions and methods that enable the integration and streamlining of the nucleic acid extraction and library preparation in a single workflow while eliminating the need for a cold chain for sample storage and transportation, wherein said compositions may comprise particles comprising an inner core loaded with lyophilized microspheres of releasable workflow reagent(s) for one-pot NGS sample preparation, and wherein said inner core is encapsulated by an outer, stimuli-responsive polymeric carrier shell that is engineered for triggered release of said lyophilized workflow reagent(s) microspheres into a biological sample in a controlled manner, in response to a specific environmental trigger or stimuli. The compositions and processes described herein provide high quality and longer gDNA strands compared to previous methods. The high quality, long DNA strands enable a high level of linked long reads in a sequencing reaction.

In accordance with the present disclosure, the compositions, systems, and methods described herein have many benefits including, for example, stabilization of reagents thus eliminating the need for cold transportation and storage, enables room-temperature shipping and storage of reagents and complete assays, protection of the encapsulated lyophilized reagent microspheres against harsh environmental conditions, time-controlled reagent release, simplifies workflows by eliminating the need to individually pipette microliter quantities of potentially expensive assay reagents, reduces the risk of sample contamination. Fewer pipetting steps and less sample handling also help minimize training requirements, reduce costs (e.g., shipping, storage, training costs) and save time. The robustness and reliability of an assay are also improved along with data quality, and the risk of sample contamination is minimized. The methods also improve data quality and reliability of results while reducing contamination risks, are compatible with downstream applications like NGS, reduce transportation costs through the ability to ship without refrigeration, increase shelf life resulting in less reagent waste, support applications in the field, including remote or poorly accessible locations with insufficient infrastructure (e.g. developing countries) without affecting sample or data quality, and provides batch-to-batch consistency with all samples treated uniformly.

### Terms

Unless defined otherwise herein, all technical and scientific terms used herein have the same meaning as commonly understood by one of ordinary skill in the art within the context of the disclosure, and in the specific context where each term is used. However, so that the present disclosure may be more readily understood, certain terms are first defined. Additional definitions are set forth throughout the application.

As used herein, the singular terms “a,” “an,” and “the” include the plural reference unless the context clearly indicates otherwise. The term “plurality” refers to more than one element. For example, the term is used herein in reference to a number of reads to produce phased island using the methods disclosed herein.

The terms “substantially”, “approximately”, “about”, “relatively”, or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, fluctuations can refer to less than or equal to +10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The term “sample” herein refers to a sample, typically derived from a biological fluid, cell, tissue, organ, or organism containing a nucleic acid or a mixture of nucleic acids containing at least one nucleic acid sequence that is to be sequenced and/or phased. Such samples include, but are not limited to sputum/oral fluid, amniotic fluid, blood, a blood fraction, fine needle biopsy samples (e.g., surgical biopsy, fine needle biopsy, etc.), urine, peritoneal fluid, pleural fluid, tissue explant, organ culture and any other tissue or cell preparation, or fraction or derivative thereof or isolated therefrom. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc. Although the sample is often taken from a human subject (e.g., patient), samples can be taken from any organism having chromosomes, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

A sample can be a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, stem cells, germ cells (e.g., sperm, oocytes), transformed cell lines and the like. For example, polynucleotide molecules may be obtained from primary cells, cell lines, freshly isolated cells or tissues, frozen cells or tissues, paraffin embedded cells or tissues, fixed cells or tissues, and/or laser dissected cells or tissues. Biological samples can be obtained from any subject or biological source including, for example, human or non-human animals, including mammals and non-mammals, vertebrates and invertebrates, and may also be any multicellular organism or single-celled organism such as a eukaryotic (including plants and algae) or prokaryotic organism, archaeon, microorganisms (e.g. bacteria, archaea, fungi, protists, viruses), and aquatic plankton.

The terms “polynucleotide,” “nucleic acid” and “nucleic acid molecules” are used interchangeably and refer to a covalently linked sequence of nucleotides (i.e., ribonucleotides for RNA and deoxyribonucleotides for DNA) in which the 3′ position of the pentose of one nucleotide is joined by a phosphodiester group to the 5′ position of the pentose of the next. The nucleotides include sequences of any form of nucleic acid, including, but not limited to RNA and DNA molecules such as cfDNA molecules. The term “polynucleotide” includes, without limitation, single- and double-stranded polynucleotide. The terms as used herein also encompasses cDNA, that is complementary, or copy DNA produced from an RNA template, for example by the action of reverse transcriptase. In one implementation, the nucleic acid to be analyzed, for example by sequencing through use of the described systems, is immobilized on a substrate (e.g., a substrate within a flow cell or one or more beads upon a substrate such as a flow cell, etc.). The term immobilized as used herein is intended to encompass direct or indirect, covalent, or non-covalent attachment, unless indicated otherwise, either explicitly or by context. The analytes (e.g., nucleic acids) may remain immobilized or attached to the support under conditions in which it is intended to use the support, such as in nucleic acid sequencing applications. In one implementation, the template polynucleotide is one of a plurality of template polynucleotides attached to a substrate. In one implementation, the plurality of template polynucleotides attached to the substrate include a cluster of copies of a library polynucleotide.

Nucleic acids include naturally occurring nucleic acids or functional analogs thereof. Particularly useful functional analogs are capable of hybridizing to a nucleic acid in a sequence specific fashion or capable of being used as a template for replication of a particular nucleotide sequence. The nucleic acid described herein can be of any length suitable for use in the provided methods. For example, the target nucleic acids can be at least 10, at least 20, at least 30, at least 40, at least 50, at least 75, at least 100, at least 150, at least 200, at least 250, at least 500, or at least 1000 kb in length or longer.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis (SBS) using reversible dye terminators, and sequencing-by-ligation.

The term “library” refers to a collection or plurality of nucleic acid template molecules which have a common use or common property such as a common origin; e.g., all members of the library come from a single sample. The members of the library may be processed or modified to so that their membership in the library is clearly identified. For example, all members of a library may share a common sequence at their 5′ ends and a common sequence at their 3′ ends. Use of the term “library” to refer to a collection or plurality of template molecules should not be taken to imply that the templates making up the library are derived from a particular source, or that the “library” has a particular composition. By way of example, use of the term “library” should not be taken to imply that the individual templates within the library must be of different nucleotide sequence or that the templates be related in terms of sequence and/or source.

The terms “address”, “index,” “index sequence,” “unique identifier,” “barcode,” “barcode sequence” and “tag” are used interchangeably herein unless specified otherwise. The terms refer to a sequence of nucleotides, usually oligonucleotides, that can be used to identify a sequence of interest such as region of a genome or haplotype. The address, index, index sequence, unique identifier, barcode, barcode sequence or tag sequence may be exogenously incorporated into the sequence of interest by ligation, extension, or other methods known in the art. The index sequence may also be endogenous to the sequence of interest, e.g., a segment in the sequence of interest itself may be used as an index. A nucleotide address, index, index sequence, unique identifier, barcode, barcode sequence or tag can be a random or a specifically designed nucleotide sequence. An address, index, index sequence, unique identifier, barcode, barcode sequence or tag can be of any desired sequence length so long as it is of sufficient length to be unique nucleotide sequence within a plurality of indices in a population and/or within a plurality of polynucleotides that are being analyzed or interrogated. A nucleotide address, index, index sequence, unique identifier, barcode, barcode sequence or tag is useful, for example, to be attached to a target polynucleotide to tag or mark a particular species for identifying all members of the tagged species within a population. Accordingly, an index is useful as a barcode where different members of the same molecular species can contain the same index and where different species within a population of different polynucleotides can have different indices.

As used herein, the term “target nucleic acid” is intended to mean a nucleic acid that is the object of an analysis or action. The analysis or action includes subjecting the nucleic acid to copying, amplification, sequencing and/or other procedure for nucleic acid interrogation. A target nucleic acid can include nucleotide sequences additional to the target sequence to be analyzed. For example, a target nucleic acid can include one or more adapters, including an adapter that functions as a primer binding site, that flank(s) a target nucleic acid sequence that is to be analyzed. A target nucleic acid hybridized to a capture oligonucleotide or capture primer can contain nucleotides that extend beyond the 5′ or 3′ end of the capture oligonucleotide in such a way that not all of the target nucleic acid is amenable to extension.

As used herein, the term “substrate” is intended to mean a solid support or support structure. The term includes any material that can serve as a solid or semi-solid foundation for creation of features such as wells for the deposition of biopolymers, including nucleic acids, polypeptide and/or other polymers. Non-limiting examples of substrates include a bead array, a spotted array, clustered particles arranged on a surface of a chip, a film, a multi-well plate, a cartridge, and a flow cell. A substrate as provided herein is modified, or can be modified, for example, to accommodate attachment of biopolymers by a variety of methods well known to those skilled in the art. Exemplary types of substrate materials include glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, a variety of polymers other than those exemplified above and multiwell microtiter plates. Specific types of exemplary plastics include acrylics, polystyrene, copolymers of styrene and other materials, polypropylene, polyethylene, polybutylene, polyurethanes and Teflon™. Specific types of exemplary silica-based materials include silicon and various forms of modified silicon.

In some implementations, the solid support comprises one or more surfaces that are accessible to contact with reagents, beads, or analytes. The surface can be substantially flat or planar. Alternatively, the surface can be rounded or contoured. Example contours that can be included on a surface are wells (e.g., microwells or nanowells), depressions, pillars, ridges, channels or the like. Example materials that can be used as a surface include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon, carbon-fiber; metal; inorganic glass; optical fiber bundle, or a variety of other polymers. A single material or mixture of several different materials can form a surface useful in certain examples. In some examples, a surface comprises wells (e.g., microwells or nanowells). In some aspects, the surface comprises wells in an array of wells (e.g., microwells or nanowells) on glass, silicon, plastic or other suitable solid supports with patterned, covalently-linked gel such as poly(N-(5-azidoacetamidylpentyl)acrylamide-coacrylamide) (PAZAM, see, for example, U.S. Pat. App. Pub. No. 2014/0079923 A1, which is incorporated herein by reference). In some examples, a support structure can include one or more layers.

As used herein, the term “plurality” is intended to mean a population of two or more different members. Pluralities can range in size from small, medium, large, to very large. The size of small plurality can range, for example, from a few members to tens of members. Medium sized pluralities can range, for example, from tens of members to about 100 members or hundreds of members. Large pluralities can range, for example, from about hundreds of members to about 1000 members, to thousands of members and up to tens of thousands of members. Very large pluralities can range, for example, from tens of thousands of members to about hundreds of thousands, a million, millions, tens of millions and up to or greater than hundreds of millions of members. Therefore, a plurality can range in size from two to well over one hundred million members as well as all sizes, as measured by the number of members, in between and greater than the above exemplary ranges. An exemplary number of features within a microarray includes a plurality of about 500,000 or more discrete features within 1.28 cm². Exemplary nucleic acid pluralities include, for example, populations of about 1×10⁵, 5×10⁵ and 1×10⁶ or more different nucleic acid species. Accordingly, the definition of the term is intended to include all integer values greater than two. An upper limit of a plurality can be set, for example, by the theoretical diversity of nucleotide sequences in a nucleic acid sample.

As used herein, the term “double-stranded,” when used in reference to a nucleic acid molecule, means that substantially all of the nucleotides in the nucleic acid molecule are hydrogen bonded to a complementary nucleotide. A partially double stranded nucleic acid can have at least 10%, 25%, 50%, 60%, 70%, 80%, 90% or 95% of its nucleotides hydrogen bonded to a complementary nucleotide.

As used herein, the term “single-stranded,” when used in reference to a nucleic acid molecule, means that essentially none of the nucleotides in the nucleic acid molecule are hydrogen bonded to a complementary nucleotide.

As used herein, the term “dNTP” refers to deoxynucleoside triphosphates. NTP refers to ribonucleotide triphosphates. The purine bases (Pu) include adenine (A), guanine (G) and derivatives and analogs thereof. The pyrimidine bases (Py) include cytosine (C), thymine (T), uracil (U) and derivatives and analogs thereof. Examples of such derivatives or analogs, by way of illustration and not limitation, are those which are modified with a reporter group, biotinylated, amine modified, radiolabeled, alkylated, and the like and also include phosphorothioate, phosphite, ring atom modified derivatives, and the like. The reporter group can be a fluorescent group such as fluorescein, a chemiluminescent group such as luminol, a terbium chelator such as N-(hydroxyethyl) ethylenediaminetriacetic acid that is capable of detection by delayed fluorescence, and the like.

As used herein, the term “size selection” means a procedure during which a subpopulation of nucleic acid fragments, majority of which have a number of nucleotides falling in a defined range, is selected from a population of nucleic acid fragments, and thus the percentage of nucleic acid fragments having a number of nucleotides falling in the defined range increases.

As used herein, the term “protease” refers to a protein, polypeptide or peptide exhibiting the ability to hydrolyze polypeptides or substrates having a polypeptide portion. The protease(s) provided in the present methods can be a single protease possessing broad specificity. The present methods can use a mixture of various proteases. The proteases provided herein can be heat-labile (i.e. thermolabile) and thus can be inactivated by heat. In certain implementations, the proteases provided herein can be inactivated at a temperature above about 35° C., 40° C., 45° C., 50° C., 55° C., 60° C., 65° C., 70° C., 75° C., 80° C. or above about 85° C. The proteases provided herein can digest chromatin proteins and other DNA-binding proteins to release naked genomic DNA, and can also digest endogenous DNase to protect DNA from degradation. The proteases provided herein include, but are not limited to, serine proteases, threonine proteases, cysteine proteases, aspartate proteases, glutamic acid proteases, and metalloproteases. Typically, aspartic, glutamic and metallo-proteases activate a water molecule which performs a nucleophilic attack on the peptide bond to hydrolyze it. Serine, threonine and cysteine proteases typically use a nucleophilic residue to perform a nucleophilic attack to covalently link the protease to the substrate protein, releasing the first half of the product. This covalent acyl-enzyme intermediate is then hydrolyzed by activated water to complete catalysis by releasing the second half of the product and regenerating the free enzyme. Exemplary protease used herein includes a serine protease isolated from a recombinant Bacillus strain. Exemplary proteases used herein include Proteinase K, subtilisin and variants thereof, including alcalase, alcalase 0.6L, alcalase 2.5L, ALK-enzyme, bacillopeptidase A, bacillopeptidase B, Bacillus subtilis alkaline proteinase bioprase, bioprase AL 15, bioprase APL 30, colistinase, subtilisin J, subtilisin S41, subtilisin Sendai, subtilisin GX, subtilisin E, subtilisin BL, genenase I, esperase, maxatase, thermoase PC 10, protease XXVII, thermoase, superase, subtilisin Carlsberg subtilisin DY, subtilopeptidase, SP 266, savinase 8.0L, savinase 4.0T, kazusase, protease VIII, opticlean, protin A 3L, savinase, savinase 16.0L, savinase 32.0 μL EX, orientase 10B, protease S, serine endopeptidase. In particular implementations of the methods and compositions presented herein, a heat-labile protease such as Proteinase K and heat-labile variants thereof can be used.

As used herein, the term “protease inhibitor” refers to a substance, e.g., a compound, capable of at least partially reducing the ability of a protease to hydrolyze peptides. Examples of protease inhibitors known in the art which can be used for the present methods include but are not limited to FOCUS™ PROTEASEARREST™ protease inhibitor cocktail, PEFABLOC® SC (4-(2-Aminoethyl)-benzolsulfonylfluorid-hydrochloride) (AEBSF) protease inhibitor, Aprotinin protease inhibitor, Bestatin protease inhibitor, Leupeptin protease inhibitor, Phenylmethylsulfonyl fluoride (PMSF) protease inhibitor, and tripeptidyl chloromethyl ketones (TCK/TPCK, TLCK, and E-64) protease inhibitors.

As used herein, the term “tagmentation” refers to the modification of DNA by a transposome complex comprising transposase enzyme complexed with adaptors comprising transposon end sequence. Tagmentation results in the simultaneous fragmentation of the DNA and ligation of the adaptors to the 5′ ends of both strands of duplex fragments. Additional sequences can be added to the ends of the adapted fragments, for example by PCR, ligation, or any other suitable methodology known to those of skill in the art. As used herein, the term “transposome complex” (TSM) refers to a transposase enzyme non-covalently bound to a double stranded nucleic acid. For example, the complex can be a transposase enzyme preincubated with double-stranded transposon DNA under conditions that support non-covalent complex formation. Double-stranded transposon DNA can include, without limitation, Tn5 DNA, a portion of Tn5 DNA (e.g., Tn5 recognition site), a transposon end composition, a mixture of transposon end compositions or other double-stranded DNAs capable of interacting with a transposase such as the hyperactive Tn5 transposase.

As used herein, the term “transposition reaction” refers to a reaction wherein one or more transposons are inserted into target nucleic acids, e.g., at random sites or almost random sites. Essential components in a transposition reaction are a transposase and DNA oligonucleotides that exhibit the nucleotide sequences of a transposon, including the transferred transposon sequence and its complement (the non-transferred transposon end sequence) as well as other components needed to form a functional transposition or transposome complex. The DNA oligonucleotides can further include additional sequences (e.g., adaptor or primer sequences) as needed or desired. In some implementations, the method provided herein is exemplified by employing a transposition complex formed by a hyperactive Tn5 transposase and a Tn5-type transposon end (Goryshin and Reznikoff, 1998, J. Biol. Chem., 273: 7367) or by a MuA transposase and a Mu transposon end comprising R1 and R2 end sequences (Mizuuchi, 1983, Cell, 35: 785; Savilahti et al., 1995, EMBO J., 14: 4893). However, any transposition system that is capable of inserting a transposon end in a random or in an almost random manner with sufficient efficiency to 5′-tag and fragment a target DNA for its intended purpose can be used in the present invention. Examples of transposition systems known in the art which can be used for the present methods include but are not limited to Staphylococcus aureus Tn552 (Colegio et al., 2001, J Bacterid., 183: 2384-8; Kirby et al., 2002, Mol Microbiol, 43: 173-86), Tyl (Devine and Boeke, 1994, Nucleic Acids Res., 22: 3765-72 and International Patent Application No. WO 95/23875), Transposon Tn7 (Craig, 1996, Science. 271: 1512; Craig, 1996, Review in: Curr Top Microbiol Immunol, 204: 27-48), TnIO and ISIO (Kleckner et al., 1996, Curr Top Microbiol Immunol, 204: 49-82), Mariner transposase (Lampe et al., 1996, EMBO J., 15: 5470-9), Tci (Plasterk, 1996, Curr Top Microbiol Immunol, 204: 125-43), P Element (Gloor, 2004, Methods Mol Biol, 260: 97-114), TnJ (Ichikawa and Ohtsubo, 1990, J Biol Chem. 265: 18829-32), bacterial insertion sequences (Ohtsubo and Sekine, 1996, Curr. Top. Microbiol. Immunol. 204:1-26), retroviruses (Brown et al., 1989, Proc Natl Acad Sci USA, 86: 2525-9), and retrotransposon of yeast (Boeke and Corces, 1989, Annu Rev Microbiol. 43: 403-34). The method for inserting a transposon end into a target sequence can be carried out in vitro using any suitable transposon system for which a suitable in vitro transposition system is available or that can be developed based on knowledge in the art. In general, a suitable in vitro transposition system for use in the methods provided herein uses, at a minimum, a transposase enzyme of sufficient purity, sufficient concentration, and sufficient in vitro transposition activity and a transposon end with which the transposase forms a functional complex with the respective transposase that is capable of catalyzing the transposition reaction. Suitable transposase transposon end sequences that can be used in the invention include but are not limited to wild-type, derivative or mutant transposon end sequences that form a complex with a transposase chosen from among a wild-type, derivative or mutant form of the transposase.

As used herein, the term “transposase” refers to an enzyme that is capable of forming a functional complex with a transposon end-containing composition (e.g., transposons, transposon ends, transposon end compositions) and catalyzing insertion or transposition of the transposon end-containing composition into the double-stranded target nucleic acid with which it is incubated, for example, in an in vitro transposition reaction. A transposase as presented herein can also include integrases from retrotransposons and retroviruses. Transposases, transposomes and transposome complexes are generally known to those of skill in the art, as exemplified by the disclosure of US 2010/0120098. Although many implementations described herein refer to Tn5 transposase and/or hyperactive Tn5 transposase, it will be appreciated that any transposition system that is capable of inserting a transposon end with sufficient efficiency to 5′-tag and fragment a target nucleic acid for its intended purpose can be used in the present invention. In particular implementations, a transposition system is capable of inserting the transposon end in a random or in an almost random manner to 5′-tag and fragment the target nucleic acid.

As used herein, the term a “library of tagged nucleic acid fragments” refers to a collection or population of tagged nucleic acid fragments (e.g., di-tagged nucleic acid fragments) generated from a resource, e.g., whole genome, wherein the combination of the tagged nucleic acid fragments in the collection or population exhibits sequences that are qualitatively and/or quantitatively representative of the sequence of the resource from which the tagged nucleic acid fragments were generated, e.g., whole genome. It is possible that a library of tagged nucleic acid fragments does not contain a tagged nucleic fragment representing every sequence which is exhibited by the resource.

As used herein, the term “primer” is an oligonucleotide (“oligo”), generally with a free 3′—OH group that can be extended by a nucleic acid polymerase. For a template-dependent polymerase, generally at least the 3 ′-portion of the primer oligo is complementary to a portion of a template nucleic acid, to which the oligo “binds” (or “complexes,” “anneals,” or “hybridizes”), by hydrogen bonding and other molecular forces, to the template to give a primer/template complex for initiation of synthesis by a DNA polymerase, and which is extended by the addition of covalently bonded bases linked at its 3 ′-end which are complementary to the template in the process of DNA synthesis. The result is a primer extension product.

As used herein, the term “adaptor” or “adapter” are used interchangeably and can refer to an oligonucleotide that may be attached to the end of a nucleic acid. Adaptor sequences may comprise, for example, priming sites, the complement of a priming site, recognition sites for endonucleases, common sequences and promoters. Adaptors may also incorporate modified nucleotides that modify the properties of the adaptor sequence. For example, phosphorothioate groups may be incorporated in one of the adaptor strands.

The compositions, systems, and methods described herein include particles having a shell surrounding a core and the core may include one or more lyophilized microspheres (i.e., the composition may include an encapsulated lyophilized microsphere).

As described herein, “encapsulate”, “encapsulated”, and “encapsulation” include the enclosing of one or more microspheres as described herein. Microencapsulation as described herein refers to the embedding of at least one ingredient, for example, an active agent, into at least one other material, for example, a shell material. Encapsulation in accordance with the present disclosure includes, but is not limited to, bulk encapsulation, macroencapsulation, microencapsulation, nanoencapsulation, single molecule, and ionic encapsulation. In accordance with the present disclosure, the compositions, systems, and methods described herein have many benefits including, for example, increasing stability of microspheres, use of macroencapsulation to enable multi-run cartridges, and use of microencapsulation to enable simplified workflows and reduced number of reagent wells. The compositions, systems, and methods described herein use encapsulation of particles that would otherwise be responsive to pH changes to stabilize these buffers and increase SBS performance.

As used herein, “microsphere” includes spherical particles that include a shell and a core and have a diameter of 0.1 μm to 1,000 μm. For example, a microsphere may have a diameter of about 0.1 μm, 0.5 μm, 1 μm, 10 μm, 20 μm, 30 μm, 40 μm, 50 μm, 60 μm, 70 μm, 80 μm, 90 μm, 100 μm, 150 μm, 200 μm, 300 μm, 400 μm, 500 μm, 600 μm, 700 μm, 800 μm, 900 μm, 1000 μm, or any diameter between about 0.1 μm and about 1,000 μm. In one implementation, the encapsulated microsphere has a diameter between about 100 μm and 1000 μm.

Microspheres may refer to lyophilized particles comprising reagents and/or active ingredients. In certain implementations, microspheres may comprise a polymer shell, for example, biodegradable polymers and/or water soluble polymers, and optionally an inner core inside the shell. Microspheres in accordance with the present disclosure include those prepared by conventional techniques, which are known to those skilled in the art. For example, microspheres may be prepared by freezing a liquid into frozen pellets, followed by placing frozen microspheres in a dryer, for example, a rotational dryer.

As described herein, a “shell” includes a composition that surrounds a core. In one implementation, a shell includes an outer layer of a microsphere and, or in the alternative, an outer layer of a macrosphere. In one implementation, the shell includes, for example, a shell material selected from the group consisting of carrageenan, agarose, poloxamer, shellac, trehalose, paraffin wax, fatty acid (myristic acid, almitic acid), and fatty acid ester, i.e. PEG stearate, gelatin, hydroxypropyl methylcellulose (HPMC), cellulose acetate, fullalin, oxygen scavenger, alginate, chitosan, starch film, benzoxaborole-poly(vinyl alcohol) (benzoxaborole-PVA), pectin, polyvinylpyrrolidone (PVP), poly(vinylpyrrolidone-co-vinyl acetate), polyvinyl alcohol (PVA), Poly(vinylalcohol-graft-PEG), one or more upper critical soluble temperature (USCT) polymers, e.g., poly(acrylamide-co-acrylonitrile), poly(N-acryloyl glycinamide), one or more lower critical soluble temperature (LCST) polymers, e.g., poly(N-isopropyl acrylamide) and its co-polymer, or any combination thereof.

As described herein, a “core” or “core region” includes any material within the surrounding shell. In various implementations, a core comprises one or more lyophilized microspheres. In various implementations, a core comprises lyophilized beads. In various implementations, a core comprises beads made of non-lyophilized sugar or plastic, optionally wherein a reagent is coated and dried on the surface of the non-lyophilized microspheres or beads. In various implementations, the core comprises one or more lyophilized beads or one or more lyophilized microspheres.

As used herein, the term “reagent” describes a single agent or a mixture of two or more agents useful for reacting with, interacting with, diluting, or adding to a sample, and may include agents used in nucleic acid reactions, including, for example buffers, chemicals, enzymes, polymerase, primers including those having a size of less than 50 base pairs, template nucleic acids, nucleotides, labels, dyes, or nucleases. A reagent as described herein may, in certain implementations, include enzymes such as polymerases, ligases, recombinases, or transposases; binding partners such as antibodies, epitopes, streptavidin, avidin, biotin, lectins or carbohydrates; or other biochemically active molecules. Other exemplary reagents include reagents for a biochemical protocol, such as a nucleic acid amplification protocol, an affinity-based assay protocol, an enzymatic assay protocol, a sequencing protocol, and/or a protocol for analyses of biological fluids. According to some implementations disclosed herein, a reagent may include one or more beads, in particular magnetic beads, depending on specific workflows and/or downstream applications.

The terms “connect,” “connected,” “contact” “coupled” and/or the like are broadly defined herein to encompass a variety of divergent arrangements and assembly techniques. These arrangements and techniques include, but are not limited to (1) the direct joining of one component and another component with no intervening components therebetween (i.e., the components are in direct physical contact); and (2) the joining of one component and another component with one or more components therebetween, provided that the one component being “connected to” or “contacting” or “coupled to” the other component is somehow in operative communication (e.g., electrically, fluidly, physically, optically, etc.) with the other component (notwithstanding the presence of one or more additional components therebetween). It is to be understood that some components that are in direct physical contact with one another may or may not be in electrical contact and/or fluid contact with one another. Moreover, two components that are electrically connected, electrically coupled, optically connected, optically coupled, fluidly connected or fluidly coupled may or may not be in direct physical contact, and one or more other components may be positioned therebetween.

### Particle Core-Shell Composite Material

The present disclosure relates to one or more particles comprising a core-shell composite materials comprising: a) an inner core optionally comprising releasable lyophilized microspheres or lyophilized beads of one or more workflow reagents; b) an outer shell encapsulating said inner core, wherein the outer shell comprises one or more layers of a stimuli-sensitive polymer(s), and wherein the outer shell is designed to be stimuli-responsive, wherein the physico-chemical properties changes upon the application of different stimuli, releasing the encapsulated lyophilized microspheres into in a specified environment (i.e., “external environment”), for example, a biological sample.

The core-shell composite material may be a macro-sized, a micro-sized or a nano-sized particle.

In one implementation, the core includes, but is not limited to, one or more reagents, for example, one or more enzyme, salt, surfactant, buffering agent, enzyme inhibitor, primer, nucleotide, organic osmolite, magnetic bead, molecular probe, crowding agent, small molecule, labelled-nucleotide, a fluorophore, or any combination thereof.

The core-shell composite may exhibit a total thickness of the shell structure of around 1-25 μm. In implementations, the thickness may be selected from 2.5, 5, 10, 15, 20, or 25 μm or the thickness may be provided in a range having an upper and lower limit selected from these values. When the outer shell comprises more than one shell layer, the said layers may be independently from 1 to 25 μm thick. In various implementations, the shell is between about 1 μM to 25 M in thickness, between about 1 μM to about 20 μM, between about 5 M to about 20 μM, between about 3 μM to about 10 μM, or between about 4 μM to about 6 M, e.g., about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 M, about 23 μM, about 24 μM, or about 25 μM.

The thickness may be advantageously adjusted according to the residence time of the composite material. For example, the shell may be at least 5 μm for a homogeneous coating which will enable predictable release.

The core-shell composite material may be substantially spherical in shape with a diameter of about 0.2 μm to about 1,000 μm. The core-shell composite material may have an average diameter of about 200 μm, 300 μm, 400 μm, 500 μm, 600 μm, 700 μm, 800 μm, 900 μm, 1000 μm. In various implementations, the microspheres, with or without coating material, have a diameter from about 300 to 700 μm, from about 350 to 625 μm, or from about 400 to 600 μm. The core-shell composite material may comprise substantially monodisperse particles, each having substantially the same average diameter. The core-shell material may also comprise lyophilized microspheres or lyophilized beads having a distribution of average diameters.

The shell, as described herein, may include one layer or a plurality of layers of varying compositions. For example, the shell may include one layer, two layers, three layers, four layers, five layers, six layers, seven layers, eight layers, nine layers, ten layers, or more than ten layers. Each of the layers may include the same or different materials from the other layers that are present in the shell.

The core-shell composite material may comprise, a shell material selected from the group consisting of hydroxypropyl methylcellulose (HPMC), Cellulose acetate, Polyethylene glycol, Poly-(Vinylpyrrolidone)-Poly-(Vinylacetate-Co-Crotonic Acid) (PVP-co-PVAc), Eudragits, Isoleucine Eudragit RL/RS, Opadry CA, polyester (i.e. Polylactic-co-glycolic acid (PLGA)), wax, UCST polymer, LSCT polymer, carrageenan, shellac, paraffin wax, fatty acid, fatty acid ester, gelatin, pullalan, oxygen scavenger, alginate, chitosan, starch film, benzoxaborole-poly(vinyl alcohol) (benzoxaborole-PVA), pectin, polyvinylpyrrolidone (PVP), polyvinyl alcohol, or any combination thereof. In one example, the shell may include, but is not limited to starch, cellulose, hydrocolloid, alginate, collagen, and any combination thereof. Water soluble (hydrophilic) polymers include ethyl cellulose (EC), methylethyl cellulose (MEC), carboxymethyl cellulose (CMC), carboxymethyl ethylcellulose (CMEC), hydroxyethyl cellulose (HEC), hydroxypropyl cellulose (HPC), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC), pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl) methacrylate chloride.

The amount of shell material includes, for example, any amount suitable to produce a desired shell result. In one implementation, the shell material is present in an amount between about 1 wt % and about 100 wt % of the shell. For example, the shell material may be present in about 1 wt %, 2 wt %, 20 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 60 wt %, 70 wt %, 80 wt %, 90 wt %, 100 wt %, of the shell, or any amount there between. In one implementation, the shell material is present in an amount between about 10 wt % and about 90 wt %, or between about 10 wt % and about 80 wt %, or between about 10 wt % and about 70 wt %, or between about 10 wt % and about 60 25 wt %, or between about 10 wt % and about 50 wt %, of the shell.

The shell as described herein, may, in various implementations, include a shell additive. The shell additive may be present in an amount between about 0.01% w/w of the shell and about 99% w/w of the shell. In one implementation, the shell additive is present in an amount between about 10%/t w/w and about 90% w/w of the shell. In one implementation, the shell additive is present in an amount between about 10% w/w and about 40% w/w. In one implementation, the shell additive is a moisture barrier material present in an amount no more than 90% w/w of the shell. In one implementation, the shell additive is present in an amount of at least 10% w/w concentration of the shell. For example, the shell additive may, in one implementation, be present in an amount between 0.1% w/w of the shell and about 15.0% w/w of the shell. For example, the shell additive may be present in an amount of about 0.01% w/w, 0.05% w/w, 0.1% w/w, 0.5% w/w, 1.0% w/w, 1.5% w/W, 2.0% w/w, 2.5% w/w, 3.0% w/w, 3.5% w/w, 4.0% w/w, 4.5% w/w, 5.0% w/w, 5.5% w/w, 6.0% w/w, 6.5% w/w, 7.0% w/w, 7.5% w/w, 8.0% w/w, 8.5% w/w, 9.0% w/w, 9.5% w/w, 10.0% w/w, 10.5% w/w, 11.0% w/w, 11.5% w/w, 12.0% w/w, 12.5% w/w, 13.0% w/w, 13.5% w/w, 14.0% w/w, 14.5% w/w, 15% w/w, or any amount therebetween. The amount of the shell additive may be adjusted to accommodate a particular reagent or combination of reagents, or to accommodate a particular microsphere composition.

Exemplary shell additives include, but are not limited to, one or more of a polymer, a copolymer, a block copolymer, an anti-static agent, an anti-foaming agent, a plasticizer, a second polyvinyl alcohol (PVA), an ammonium salt, a conductivity promoter, a stearate derivative, an oleate derivative, a laurate derivative, a polyether compound, an amino acid, tocopherol acetate, piperidyl sebacate, sodium salt, a buffer, a chelating agent, imidazolium salt, polyaniline, or any combination thereof. In one implementation, the polyether compound is selected from polyethylene glycol, polypropylene glycol, a block copolymer derived from ethylene oxide (EO) and propylene oxide (PO), or any combination thereof. In one implementation, the stearate derivative or oleate derivative is selected from magnesium stearate, PEG stearate, triglycerol stearate. Span® 60, Tween® 60, glycerol trioleate, Tween® 80, or any combination thereof. In some implementations, the amino acid is selected from one or more of leucine, isoleucine, phenylalanine, or any combination thereof. In one implementation, the polymer is neutral, cationic, or anionic. In some implementations, the sodium salt is selected from one or more of sodium chloride, sodium bisulfite, sodium citrate, or any combination thereof. In various implementations, the buffer is Trizma, Tris. HCl, or a combination thereof. In one implementation, the ammonium salt is selected from tetraalkyl ammonium chloride, tris(hydroxyethyl) alkylammonium chloride, or a combination thereof. In one implementation, the imidazolium salt is selected from 1-ethyl-3-methyl-imidazolium salt or polyquaternium or Luviquat® (copolymer of vinyl pyrrolidone and quaternized vinylimidazole) or a combination thereof. In one implementation, the shell additive comprises ammonium salt, copolymer, polyvinyl alcohol graft polyethylene glycol copolymer, polyvinyl alcohol (PVA), or any combination thereof. In various implementations, the shell additive is magnesium stearate or polyethylene glycol stearate.

### The Inner Core and Lyophilized Microspheres

As described herein, a “core” or “core region” includes any material within the encapsulating shell.

A core in accordance with the present disclosure comprises one or more lyophilized microspheres or lyophilized beads.

The lyophilized microspheres of the present disclosure can comprise any reagent that is desired for controlled delivery and that can be unitized in substantially small sizes to be amenable to being lyophilized or particularized in size ranges described herein.

In some implementations, the inner core comprises lyophilized reagents that are suitable for use in multiple sequential co-assays comprising lysis, DNA analysis, RNA analysis, protein analysis, tagmentation, nucleic acid amplification, nucleic acid sequencing, DNA library preparation, SBS technology, assay for transposase accessible chromatic using sequencing (ATAC-seq), contiguity-preserving transposition (CPT-seq), single cell combinatorial indexed sequencing (SCI-seq), or single cell genome amplification, or any combination thereof performed sequentially. In one implementation, the composition is used for performing multiple co-assay reactions. The compositions, systems, and methods described herein (e.g., encapsulation of lyophilized microspheres) may, in one implementation, improve sequencing quality, enable one-pot library prep, and simplify manufacturing. As used herein, the term “one-pot reaction” may also be referred to as “transfer-free reaction.”

In further implementations, the inner core comprises lyophilized reagents that may be prepared for various stages of sequencing including, but not limited to, sample extraction, library preparation, enrichment, clustering, and sequencing.

### Lyophilized Spheres Comprising Sample Preparation Reagents

In one implementation, the lyophilized microspheres comprise lyophilized lysis solution. In one implementation, a lyophilized cake comprises lyophilized lysis solution. A lysis solution enables efficient lysis (e.g., of cells in a biological sample) to release nucleic acids, effectively protects the released nucleic acids from degradation in the lysate by inhibiting or degrading nucleases, and is compatible with subsequent steps for analysis of the extracted nucleic acids, such as target capture, amplification, detection, and/or sequencing. The components of the lysis buffer can be tailored depending on the types and source of cells, the desired final molecule or structure, and the level of their functionality.

In one implementation, the lyophilized microspheres comprise a lysis buffer for DNA extraction from whole blood. In one implementation, a lyophilized cake comprises a lysis buffer for DNA extraction from whole blood. Whole blood and blood fractions are a common biological starting sample for DNA extraction, for example in most epidemiologic studies. Compared to other minimally invasive sources of genomic gDNA (gDNA), such as saliva or buccal cells, gDNA yield from blood or blood fractions is comparatively higher and less fragmented (Koshy et al., Mol Biol Rep. 44(1):97-108, 2017). Whole blood contains red blood cells (RBCs), nucleated white blood cells (WBCs), platelets, and plasma. Genomic DNA is found in the nuclei of WBCs. Unlike the WBCs, mature RBCs are nonnucleated and therefore do not contain DNA. Most DNA extraction procedures from whole blood comprise a two-step lysis approach: Step 1; selective lysis and removal of RBCs with minimal effect on WBCs. RBCs contain no DNA and are a potential source of downstream inhibitors. Thus it can be advantageous to separate them from WBCs prior to DNA isolation. Lysis of WBCs to extract DNA and degrade proteins, followed by DNA recovery and washing is also contemplated.

In various implementations, a lyophilized lysis solution of the present disclosure contains a buffer (such as Tris-HCl), a broad-spectrum protease (such as Proteinase K), an amphiphilic reagent (such as a detergent, or surfactant, or a mixture thereof), chelating reagents (such as EDTA or CDTA), and a lyoprotectant/lyophilization reagent (such as sucrose or trehalose).

In one implementation, the lyophilized microspheres of the present disclosure provide reagents for a passive, one-step whole blood lysis approach using a lysing buffer mix capable of lysing both WBCs and RBCs cell types in one step. This one-step lysis approach has a number of advantages over the traditional two-step lysis method including: improved DNA yield due to elimination of sample loss incurred in a two-step procedure, single-vessel reaction which eliminates the need for pipetting, which also lowers the risk of contamination, saves time and lowers reagent cost for additional enzymes e.g., RNase.

A first component of the lysis solution is a buffer that maintains the pH of the solution (e.g., a Tris buffer or any known buffer). For example, the pH of the buffer may be at least about 8, at least about 8.5, or even at least about 9 (e.g., 8.1, 8.4, 8.6, 8.7, 8.9, 9.1, or 9.5). The buffer may have a pKa of at least about 8 (e.g., 8.1, 8.3, 8.5, 8.6, 8.8, or 8.9), and may be used at a concentration of 50-150 mM (e.g., 60 mM, 70 mM, 80 mM, 90 mM, 100 mM, 120 mM, or 140 mM). In some implementations, Tris buffer is an appropriate buffer. In some instances, Tris buffer with a pH of 8.0 and a concentration of 100 mM is used. In some other implementations, a base may be used to adjust the pH of the lysis solution. The base may be one that can raise the pH of the solutions to no less than 7 (e.g., pH 7.5, 8, 8.5, or 9.0). In some instances, the base may be an alkali-metal hydroxide. Such alkali-metal hydroxides include, but not limited to, sodium hydroxide, potassium hydroxide, and lithium hydroxide.

In some implementations the lysis solution comprises a broad-spectrum protease for proteolytic lysis. In some implementations, the broad-spectrum proteases comprise a serine protease, a threonine protease, a cysteine protease, an aspartate protease, a glutamic acid protease, or a metalloprotease. In some implementations, the broad-spectrum protease is a serine protease. In some implementations, the serine protease is Proteinase K. Proteinase K is a stable serine protease that is active under a wide range of pH, temperature, salt, solvent, and detergent concentrations. The activity of Proteinase K peaks in the presence of moderate denaturants, 2-4 molar chaotropic salts and ionic detergents, which act both to stimulate enzymatic activity and increase substrate accessibility by destabilizing protein secondary structure. At completion, Proteinase K digestion will have reduced polypeptides to small di- and tri-peptides, and in the process degraded itself by autodigestion, thus eliminating the vast majority of enzyme added to samples. Proteolysis buffer is a key additive in DNA extraction methods, and critical to DNA isolation from complex biological samples. In sample mixtures, proteolysis buffer is designed to preserve target nucleic acids, establish optimum conditions for proteolysis, solubilize lipids and microvesicles, breakdown colloids and particulate matter, and inhibit or prevent precipitation over the course of protease reactions.

Proteinase K may be present in the lysis buffer at a concentration of about 0.001 mg/mL to about 10 mg/mL. For example, the concentration of proteinase K in the lysis buffer may be about 0.001 mg/mL, about 0.005 mg/mL, about 0.01 mg/mL, about 0.05 mg/mL, about 0.1 mg/mL, about 0.5 mg/mL, about 1 mg/mL, about 1.5 mg/ml, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL or greater than about 10 mg/mL. In certain instances, a suitable Proteinase K Solution has a concentration of 20 mg/mL Proteinase K. In some embodiments, a suitable lysis solution comprises Proteinase K at a concentration of about 0.45 to about 1.8 mg/mL. In some embodiments, a suitable lysis solution comprises Proteinase K at a concentration of about 0.8 mg/mL.

If nucleic acid preparation and tagmentation steps are performed in the same reaction tube, it can be beneficial that the proteases according to the present method can be effectively inactivated without disturbing the next tagmentation step which typically uses double-stranded DNA. In some implementations, the proteases can be inactivated by increasing temperature prior to the tagmentation step. High temperature can denature double-stranded DNA conformation. Thus, in some implementations, the proteases provided herein can be inactivated at relatively low temperature without denaturing double-stranded DNA. In some implementations, one or more proteases are inactivated by increasing temperature to 50° C.-80° C. In some implementations, the one or more proteases are inactivated by increasing temperature to 50° C., 55° C., 60° C., 65° C., 70° C., 75° C. or 80° C. In various implementations, the protease is Proteinase K that can be heat inactivated.

In some implementations, the lysis solution comprises a detergent. Detergents can act as both a lysing agent and as an inhibitor of analyte degradation following the lysis of blood cells. Detergents are particularly useful for inhibiting the degradation of nucleic acids. Non-limiting examples of surfactants or detergents that may be used include: Non-ionic surfactants including polyoxy ethylene glycol alkyl ethers (sold as Brij® series detergents including Brij® 58, Brij® 52, Brij® L4 and Brij® L23), octaethylene glycol monododecyl ether, pentaethylene glycol monododecyl ether, polyoxypropylene glycol alkyl ethers, glucoside alkyl ethers (e.g., decyl glucoside, lauryl glucoside, octyl glucoside), polyoxyethylene glycol octylphenol ethers (e.g., Triton X-100), polyoxyethylene glycol alkylphenol ethers (e.g., nonoxynol-9), glycerol alkyl esters (e.g., glyceryl laurate), polyoxyethylene glycol sorbitan alkyl esters (e.g., polyoxyethylene glycol (20) sorbitan monolaurate (TWEEN® 20), polyoxyethylene glycol (40) sorbitan monolaurate (TWEEN® 40), polyoxyethylene glycol (20) sorbitan monopalmitate, polyoxyethylene glycol (20) sorbitan monostearate, polyoxyethylene glycol (4) sorbitan monostearate, polyoxyethylene glycol (20) sorbitan tristearate, polyoxyethylene glycol (20) sorbitan monooleate)), sorbitan alkyl esters (e.g., sorbitan monolaurate, sorbitan monopalmitate, sorbitan monostearate, sorbitan monooleate, sorbitan sesquioleate, sorbitan trioleate, sorbitan isostearate), cocamide monoethanolamine, cocamide diethanolamine, dodecyldimethylamine oxide, poloxamers including those sold under the Pluronic®, Synperonic® and Kolliphor® tradenames, and polyethoxylated tallow amine (POEA); Anionic surfactants including ammonium lauryl sulfate, ammonium perfluorononanoate, docusate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate (SDS), sodium dodecylbenzenesulfonate, sodium laurate, sodium lauryl ether sulfate, sodium lauroyl sarcosinate, sodium myreth sulfate, sodium pareth sulfate, sodium stearate; cationic surfactants including benzalkonium chloride, benzethonium chloride, bronidox, cetrimonium bromide, cetrimonium chloride, distearyldimethylammonium chloride, lauryl methyl gluceth-10 hydroxypropyl dimonium chloride, octenidine dihydrochloride, olaflur, and tetramethylammonium hydroxide; and Zwitterionic surfactants including CHAPS detergent, cocamidopropyl betaine, cocamidopropyl hydroxysultaine, dipalmitoylphosphatidylcholine, lecithin, hydroxysultaine, and sodium lauroamphoacetate.

Although anionic, cationic, and zwitterionic detergents may all be used in a lysis solution, the lysis solution of the present disclosure comprises at least one anionic surfactant and at least one non-ionic surfactant. In one implementation, the lysis solution contains the anionic surfactant SDS and the non-ionic surfactant TWEEN® 20.

In one implementation, the SDS may be present at a concentration of about 0.1% to about 10% (weight/volume). For example, suitable SDS concentrations include, but are not limited to, from about 0.1% to about 0.2%, from about 0.2% to about 0.3%, from about 0.3% to about 0.4%, from about 0.4% to about 0.5%, from about 0.5% to about 0.6%, from about 0.6% to about 0.7%, from about 0.7% to about 0.8%, from about 0.8% to about 0.9%, from about 0.9% to about 1%, from about 1% to about 2%, from about 2% to about 3%, from about 3% to about 4%, from about 4% to about 5%, from about 5% to about 6%, from about 6% to about 7%, from about 7% to about 8%, from about 8% to about 9%, and from about 9% to about 10%, as well as combinations of the above ranges. such as about 0.1% to about 0.5%, about 1% to about 2%, about 1% to about 5%, about 3% to about 7%, about 5% to about 9%, about 1%, about 2%, about 3%, about 4%, about 5%, about 6%, about 7%, about 8%, about 9%, or about 10%.

In one implementation, the TWEEN® 20 may be present at a concentration about 0.5% to about 10% (weight/volume percent). For example, suitable TWEEN® 20 concentrations include, but are not limited to, from about from about 0.5% to about 0.6%, from about 0.6% to about 0.7%, from about 0.7% to about 0.8%, from about 0.8% to about 0.9%, from about 0.9% to about 1%, from about 1% to about 2%, from about 2% to about 3%, from about 3% to about 4%, from about 4% to about 5%, from about 5% to about 6%, from about 6% to about 7%, from about 7% to about 8%, from about 8% to about 9%, and from about 9% to about 10%, as well as combinations of the above ranges. such as about 0.1% to about 0.5%, about 1% to about 2%, about 1% to about 5%, about 3% to about 7%, about 5% to about 9%, about 1%, about 2%, about 3%, about 4%, about 5%, about 6%, about 7%, about 8%, about 9%, or about 10%.

Alternatively, the concentration of the surfactant is measured in mg/ml or in g/L. In typical embodiments, either surfactant is present at about 1-5 mg/ml, at about 5-10 mg/ml, at about 10-15 mg/ml, at about 15-25 mg/ml, at about 25-50 mg/ml, at about 50-60 mg/ml, at about 60-70 mg/ml, at about 70-80 mg/ml, and at about 80 to 90 mg/ml, as well as combinations of the above ranges.

In order to reduce or prevent degradation of nucleic acids nuclease-free water is used in the lysis solution. In some implementations, a chelating agent also may be used to inhibit or prevent degradation of contaminating nucleic acid. The use of a chelating agent inhibits or prevents nucleic acid polymers from being degraded to smaller fragments, which may cause additional contamination problems. The chelating agent may be present at a concentration of 1-100 mM (e.g., 2 mM, 5 mM, 8 mM, 10 mM, 15 mM, 20 mM, 25 mM, 35 mM, 45 mM, 50 mM, 65 mM, 75 mM, 85 mM, or 95 mM), or at a concentration of 1-10 mM (e.g., 1.5 mM, 2 mM, 3 mM, 4 mM, 6 mM, 7 mM, or 9 mM). In some instances the chelating agent ethylenediamine tetraacetic acid (EDTA) is used. In other instances, the chelating agent cyclohexane-N,N,N′,N′ tetraacetic acid (CDTA) is used.

An anti-coagulant, if present in the lysis reagent, is at a concentration sufficient to inhibit clotting of the sample (e.g., whole blood or red blood cells). By inhibiting clotting, the anti-coagulant eliminates the need to centrifuge samples during the method to isolate red blood cells. Exemplary anti-coagulants include EDTA, EDTA-Na₂, ethylene glycol-bis(β-aminoethyl ether)-N,N,N′, N′-tetraacetic acid (EGTA), heparin, or citrate. Exemplary concentrations of EDTA in the lysis reagent include from about 0.05 mM to about 15 mM, from about 0.1 mM to about 10 mM, from about 0.5 mM to about 5 mM, about 10 mM, about 2.5 mM or about 0.1 mM. Exemplary concentrations of EDTA-Na₂ in the lysis reagent include from about 0.05 mM to about 15 mM, from about 0.1 mM to about 10 mM, from about 0.5 mM to about 5 mM, about 10 mM, about 2.5 mM, or about 0.1 mM.

In further implementations, the lysis solution also contains cryoprotective agents (CPAs) or cryoprotectants. Cryoprotectants (which may be also called excipients or cryoprotective agents, lyoprotectants or lyophilization reagent) contribute to the preservation of the structures of proteins, liposome bilayers and other substances during freezing in general. Lyoprotectants stabilize these substances during drying, especially freeze-drying. In freeze-drying lyoprotectant may be also considered as a cryoprotectant, so as used herein the term “cryo-protectant” may also include lyoprotectants. Protective additives can be generally considered to have two types: (i) amorphous glass forming, and (ii) eutectic crystallizing salts. Examples of lyoprotectants include polyhydroxy compounds such as sugars (mono-, di-, and polysaccharides), trehalose and sucrose as natural lyoprotectants and polyalcohols, such as glycerol, mannitol, sorbitol, and their derivatives. Both of these groups belong to the type (i).

In one implementation the cryoprotective and/or lyoprotective agent(s) is selected from the group consisting of trehalose, sucrose, mannitol, maltose, maltodextrin, dextran, inulin, and raffinose. In one implementation the cryoprotectant is trehalose. Trehalose, also known as α, α-trehalose; a-D-glucopyranosyl-(1→1)-a-D-gluco-pyranoside, mycose or tremalose, is a natural alpha-linked disaccharide formed by an a, a-1,1-glucoside bond between two a-glucose units. Trehalose may be present as anhydrous or as dihydrate. In one implementation the trehalose is D(+)-trehalose dehydrate.

The trehalose concentration can be measured in in mg/ml or in g/L. In typical embodiments, trehalose is present at about 5-250 mg/ml. For example, suitable trehalose concentrations include, but are not limited to, from about 5 mg/mL to about 75 mg/mL, from about 50 mg/mL to about 200 mg/mL; from about 75 mg/mL to about 200 mg/mL; from about 100 mg/mL to about 200 mg/mL; from about 25 mg/mL to about 175 mg/mL; from about 50 mg/mL to about 175 mg/mL; from about 75 mg/mL to about 175 mg/mL; from about 100 mg/mL to about 175 mg/mL; from about 25 mg/mL to about 150 mg/mL; from about 50 mg/mL to about 150 mg/mL; from about 75 mg/mL to about 150 mg/mL; from about 100 mg/mL to about 150 mg/mL; from about 25 mg/mL to about 125 mg/ml; from about 50 mg/mL to about 125 mg/mL; from about 75 mg/mL to about 125 mg/ml; from about 25 mg/mL to about 100 mg/mL, from about 125 mg/mL to about 175 mg/mL, from about 125 mg/mL to about 200 mg/mL, from about 5 mg/mL to 200 mg/mL, from about 200 mg/mL to 250 mg/mL, from about 5 mg/mL to 250 mg/mL, from about 75 mg/mL to 250 mg/mL, from about 100 mg/mL to 250 mg/mL, or from about 150 mg/mL to 250 mg/mL. In various implementations, the trehalose is in a concentration of about 150 mg/ml.

In various implementations, the method further comprises a step of de-escalating the sample to reduce or eliminate active virus in the sample. De-escalation includes, but is not limited to, guanidinium sequestration, heat inactivation (e.g., between about 55° C. to about 80° C.) with lysis buffer, addition of detergents (e.g. Triton X-100, SDS, Tween 20, Brij, ChAPS), addition of chelators (e.g. EDTA), or degrading enzymes (e.g., Proteinase K).

An example of a lysis buffer contemplated herein is set out in Table 1.

TABLE 1
Components and concentrations of an exemplary 2X lysis buffer

| Component | Reagent | Function | Conc. | | mg/ml |
|---|---|---|---|---|---|
| Anionic | Sodium Dodecyl Sulfate | Cell and nuclear | 0.40% | | 10 |
| surfactant | (SDS) | membrane | | | |
| | | disruption, Protein | | | |
| | | denaturation | | | |
| Buffering agent | Tris HCl (pH 8) | pH maintenance | 100 | mM | 12 |
| Chelating agent | Ethylenediaminetetraacetic | Inactivation of | 2 | mM | 0.58 |
| | acid (EDTA) | nucleases; | | | |
| | | anticoagulant | | | |
| Nonionic | TWEEN ® 20 | Sequestration of | 1% | | 25 |
| surfactant | | SDS, thereby | | | |
| | | mitigating its | | | |
| | | inhibitory effect on | | | |
| | | downstream | | | |
| | | enzymatic processes | | | |
| Broad spectrum | Proteinase K | Cleaves | 0.056 | mM | 1.6 |
| proteolytic | | glycoproteins and | | | |
| enzyme | | inactivates RNases | | | |
| | | and DNases. | | | |
| Cryoprotectant/ | Trehalose | Stabilization of | 400 | mM | 150 |
| lyoprotectant | | Proteinase K during | | | |
| | | lyophilization | | | |
| Total mass | | | | | 199.93 |
| Total solute | | | | | 19.99 |
| content (%) | | | | | |

### Tagmentation Solution

In one implementation the lyophilized spheres comprise a lyophilized tagmentation solution, suitable for carrying out an in vitro transposition reaction as described herein, e.g., comprising a transposase, DNA oligonucleotides that exhibit the nucleotide sequences of a transposon, components needed to form a functional transposition or transposome complex, adapter and/or primer sequences.

In some implementations, the disclosure provides a nucleic acid fragmentation reaction mixture comprising: (i) a plurality of transposases, (ii) a polynucleotide containing a first transposon end sequence, (iii) a polynucleotide containing a second transposon end sequence, (iv) a target nucleic acid molecule, and (v) an activating cation.

In some implementations, the first transposon end sequence is capable of binding to the plurality of transposases. In some implementations, the first transposon end sequence contains at least one modification, including a lesion such as a nick, gap, apurinic site or apyrimidinic site.

In some implementations, the second transposon end sequence is capable of binding to the plurality of transposases.

In some implementations, the second transposon end sequence contains at least one modification, including a lesion such as a nick, gap, apurinic site or apyrimidinic site.

In some implementations, the first and the second transposon end sequences contain at least one modification, including a lesion such as a nick, gap, apurinic site or apyrimidinic site.

In some implementations, the first or the second transposon end sequence lacks a modification, including a lesion such as a nick, gap, apurinic site or apyrimidinic site.

In some implementations, the first and the second transposon end sequences have identical or different sequences.

In some implementations, the nucleic acid fragmentation reaction mixture comprises: (i) a plurality of transposases, (ii) a polynucleotide containing a first transposon end sequence, wherein the first transposon end sequence is capable of binding to the plurality of transposases and wherein the first transposon end sequence contains at least one modification, including a lesion such as a nick, gap, apurinic site or apyrimidinic site, (iii) a polynucleotide containing a second transposon end sequence, wherein the second transposon end sequence is capable of binding to the plurality of transposases and wherein the second transposon end sequence contains at least one modification, including a lesion such as a nick, gap, apurinic site or apyrimidinic site, (iv) a target nucleic acid molecule, and (v) an activating cation (e.g., magnesium or manganese). In some implementations, the target nucleic acid molecule comprises a target DNA molecule.

In some implementations, the nucleic acid fragmentation reaction mixture further comprises a buffer (e.g., Tris-Acetate), a Proteinase K inhibitor, an SDS chelator (e.g. alpha-or beta-cyclodextrin), and an activating cation. An activating cation includes any cation required by a transposase for catalyzing a transposition reaction (e.g., magnesium —magnesium acetate).

In various implementations, the one or more tagmentation reagents comprises a bead linked transposomes (BLT), Proteinase K inhibitor, random hexamers, primers, probes, transposase, buffers, divalent cations, Tris buffer, cobalt buffer, and/or a lyophilization reagent.

While any buffer suitable for the used transposase may be used in the methods of the present invention, it is preferred to use a buffer particularly suitable for efficient enzymatic reaction of the used transposase. In this regard, a buffer comprising dimethylformamide is particularly preferred for use in the methods of the present invention, in particular during the transposase reaction. In addition, buffers comprising alternative buffering systems including TAPS, Tris-acetate or similar systems can be used. Moreover, crowding reagents as polyethylenglycol (PEG) are useful to increase tagmentation efficiency of very low amounts of DNA. Conditions for the tagmentation reaction are described by Picelli et al. (2014) Genome Res. 24:2033-2040.

A transposase contemplated within the context of the present invention is Transposase (Tnp) Tn5. Tn5 is a member of the RNase superfamily of proteins which includes retroviral integrases. Tn5 can be found in Shewanella and Escherichia bacteria. The transposon codes for antibiotic resistance to kanamycin and other aminoglycoside antibiotics. Tn5 and other transposases are notably inactive. Because DNA transposition events are inherently mutagenic, the low activity of transposases is beneficial to reduce the risk of causing a fatal mutation in the host, and thus eliminating the transposable element. One of the reasons Tn5 is so unreactive is because the N- and C-termini are located in relatively close proximity to one another and tend to inhibit each other. This was elucidated by the characterization of several mutations which resulted in hyperactive forms of transposases. One such mutation, L372P, is a mutation of amino acid 372 in the Tn5 transposase. This amino acid is generally a leucine residue in the middle of an alpha helix. When this leucine is replaced with a proline residue the alpha helix is broken, introducing a conformational change to the C-Terminal domain, separating it from the N-Terminal domain enough to promote higher activity of the protein. Accordingly, it is preferred that such a modified transposase be used, which has a higher activity than the naturally occurring Tn5 transposase. In addition, it is particularly preferred that the transposase employed in the methods of the invention is loaded with oligonucleotides, which are inserted into the target nucleic acid, in particular the target DNA.

Accordingly, use of a hyperactive Tn5 transposase and a Tn5-type transposase recognition site are contemplated (Goryshin and Reznikoff, J. Biol. Chem., 273:7367 (1998)), or MuA transposase and a Mu transposase recognition site comprising R1 and R2 end sequences (Mizuuchi, K., Cell, 35: 785, 1983; Savilahti, H, et al, EMBO J., 14: 4893, 1995). More examples of transposition systems that can be used in the methods of the present invention include Staphylococcus aureus Tn552 (Colegio et al, J. Bacteriol, 183: 2384-8, 2001; Kirby C et al, Mol. Microbiol, 43: 173-86, 2002), Tyl (Devine & Boeke, Nucleic Acids Res., 22: 3765-72, 1994 and International Publication WO 95/23875), Transposon Tn7 (Craig, NL, Science. 271: 1512, 1996; Craig, N L, Review in: Curr Top Microbiol Immunol, 204:27-48, 1996), Tn/O and IS 10 (Kleckner N, et al, Curr Top Microbiol Immunol, 204:49-82, 1996), Mariner transposase (Lampe D J, et al, EMBO J., 15: 5470-9, 1996), Tel (Plasterk R H, Curr. Topics Microbiol. Immunol, 204: 125-43, 1996), P Element (Gloor, G B, Methods Mol. Biol, 260: 97-1 14, 2004), Tn3 (Ichikawa & Ohtsubo, J Biol. Chem. 265:18829-32, 1990), bacterial insertion sequences (Ohtsubo & Sekine, Curr. Top. Microbiol. Immunol. 204: 1-26, 1996), retroviruses (Brown, et al, Proc Natl Acad Sci USA, 86:2525-9, 1989), and retrotransposon of yeast (Boeke & Corces, Annu Rev Microbiol. 43:403-34, 1989). More examples include IS5, TnIO, Tn903, IS91 1, and engineered versions of transposase family enzymes (Zhang et al, (2009) PLOS Genet. 5:e1000689. Epub 2009 Oct. 16; Wilson C. et al (2007) J. Microbiol. Methods 71:332-5) and those described in U.S. Pat. Nos. 5,925,545; 5,965,443; 6,437,109; 6,159,736; 6,406,896; 7,083,980; 7,316,903; 7,608,434; 6,294,385; 7,067,644, 7,527,966; and International Patent Publication No. WO2012103545, all of which are specifically incorporated herein by reference in their entirety.

The transposase enzyme catalyzes the insertion of a nucleic acid, in particular a DNA in a target nucleic acid, in particular target DNA. The target nucleic acid, in particular target DNA, for insertion is comprised in the isolated chromatin bound by the agent binding to chromatin. The transposase used in the methods of the present disclosure is loaded with oligonucleotides, which are inserted into the target nucleic acid, in particular the target DNA. The complex of transposase and oligonucleotide is also referred to as a transposome. In various implementations, the transposome is a heterodimer comprising two different oligonucleotides for integration. In this regard, the oligonucleotides that are loaded onto the transposase comprise multiple sequences. In particular, the oligonucleotides comprise, at least, a first sequence and a second sequence. The first sequence is used for loading the oligonucleotide onto the transposase. Exemplary sequences for loading the oligonucleotide onto the transposase are given in US 2010/0120098. The second sequence comprises a linker sequence used for primer binding during amplification, in particular during PCR amplification. Accordingly, the oligonucleotide comprising the first and second sequence is inserted in the target nucleic acid, in particular the target DNA, by the transposase enzyme. The oligonucleotide may further comprise sequences comprising barcode sequences. Barcode sequences may be random sequences or defined sequences. In this regard, the term “random sequence” in accordance with the invention is to be understood as a sequence of nucleotides, wherein each position has an independent and equal probability of being any nucleotide. The random nucleotides can be any of the nucleotides, for example G, A, C, T, U, or chemical analogs thereof, in any order, wherein: G is understood to represent guanylic nucleotides, A adenylic nucleotides, T thymidylic nucleotides, C cytidylic nucleotides and U uracylic nucleotides. The skilled person will appreciate that known oligonucleotide synthesis methods may inherently lead to unequal representation of nucleotides G, A, C, T or U. For example, synthesis may lead to an overrepresentation of nucleotides, such as G in randomized DNA sequences. This may lead to a reduced number of unique random sequences as expected based on an equal representation of nucleotides. The oligonucleotide for insertion into the target nucleic acid, in particular DNA, may further comprise sequencing adaptors, for example adaptors suitable for nanopore sequencing or Roche 454 sequencing. Furthermore, the oligonucleotide may comprise biotin tag sequences. It is preferred that the oligonucleotide loaded onto the transposase comprises said first and second sequence and a barcode sequence for indexing. Integration of barcode sequences during the transposase reaction allows the unique identification of each nucleic acid fragment, in particular DNA fragment, during sequencing analysis and/or mapping of molecular interactions.

The time required for the used transposase to efficiently integrate a nucleic acid, in particular a DNA, in a target nucleic acid, in particular target DNA, can vary depending on various parameters, like buffer components, temperature and the like. Accordingly, various incubation times may be tested/applied before an optimal incubation time is found. Optimal in this regard refers to the optimal time taking into account integration efficiency and/or required time for performing the methods of the invention. While varying incubation times are not necessarily correlated to efficient integration of said nucleic acid, in particular said DNA, in said target nucleic acid, in particular target DNA, it is preferred to use incubation times of less than 10 minutes, less than 5 minutes, less than 2 minutes, or 1 minute. Furthermore, parameters like temperature and volume may be altered for best yield. In this regard, the recommended incubation temperature for Tn5 transposase is about 37° C. Therefore, the methods herein comprise a step of addition of transposase and subsequently incubation for tagmentation at about 37° C., optionally for about 1 to 5 min. However, alternative reaction temperatures may also be employed, e.g., temperatures above about 16° C. and below about 55° C. are used in order to maintain sample integrity and transposase efficiency.

The methods for preparing a sequencing library may further comprise an amplification step for integrating said adaptor sequences. Amplification is done as described below. The adaptor sequences vary depending on the sequencing method used subsequent to preparing the sequencing library. For example, where Illumina sequencing is used, i5 and i7 ends may be attached to the nucleic acid fragments. This may also be achieved by the transposase reaction where oligonucleotides loaded onto the transposase enzyme comprise sequencing compatible adaptor sequences.

Primers suitable for use in the methods comprise sequences hybridizable to the second sequence comprised in the oligonucleotides comprised in the transposomes used in the methods of the invention. In addition, primers may comprise sequences used for sequencing. In various implementations, specific primers are used that are compatible with the subsequently used sequencing method. In this regard, Illumina sequencing, as one method of sequencing, is compatible with primers introducing flowcell ends, which can hybridize to the flowcell needed in cluster amplification. In this regard, primers may introduce i5 and i7 ends for Illumina sequencing. Furthermore, primers may introduce barcodes for multiplexing. In particular, barcodes comprised in the primer sequences may be used as unique molecular identifiers to discriminate between PCR duplicates and/or as defined barcodes to combine multiple experiments in one sequencing run.

TABLE 2
Components and concentrations of
an exemplary 2X Sample buffer

| Component | Conc. | | mg/ml |
|---|---|---|---|
| Tris Acetate | 20 | mM | 3.6238 |
| Magnesium Acetate | 10 | mM | 1.4239 |
| α-Cyclodextrin | 5.4% (w/v) | | 0.58 |
| Trehalose | 400 | mM | 136.9184 |
| optionally, a | 0.056 | mM | 1.6 |
| Proteinase K inhibitor | | | |

| | |
|---|---|
| Total mass | 195.97 |
| Total solute content (%) | 19.60 |

### Trigger Release Mechanisms

The polymeric shells are configured to resist breakdown or degradation for a time so that delivery of the core can be delayed as desired. The polymer imparts chemical and/or mechanical properties to the particle such that the core's contents, e.g., lyophilized microspheres or beads, can be released substantially only at the desired time after delivery. For example, the polymer or polymer composite can be configured for degradation under one or more conditions (two trigger mechanism), and the contents in the core can be released from the particle when the shell at least partially degrades. In one or more implementations, degradation can proceed via one or more of thermal degradation, oxidative degradation, chemical degradation, photodegradation, pressure-dependent degradation, ultrasonic degradation, and mechanical degradation.

In order to provide control of the degradation, the shell can be formed so as to include one or more chemical functionalities. In one non-limiting example, the shell can include polymers that thermally degrade (e.g., at a desired high or low temperature range) such as polyesters, polyurethanes, polyamides, poly(dialkyl siloxanes), and polycarbonates. In one non-limiting example, the vehicle can contain a thermal labile group, such as an azo compound, that degrades at a defined temperature. The shell can be configured such thermal degradation proceeds at a temperature of about 40° C. or greater, about 50° C. or greater, about 60° C. or greater, about 70° C. or greater, or about 80° C. or greater. In alternate implementations, the shell comprises polymers that solubilize under a trigger temperature, for example below 15° C.

In one or more implementations, the shell can be configured to remain substantially intact at the point of delivery; however, the shell can be further configured to release the reagents in the core, e.g., lyophilized microspheres or beads, over time. As a non-limiting example, the shell can be configured so that release of the core is delayed for a specific period of time. In one or more implementations, core release may be substantially absent under standard conditions (e.g., up to a minimum temperature, such as up to about 20° C., 25° C., 30° C., 35° C., 40° C., up to about 50° C., or up to about 60° C.), but release could be triggered when such standard conditions are exceeded. As non-limiting examples, by altering the polymer crosslink density, hydrophobic/hydrophilic balance, particle size, shell thickness and thermal properties, and/or ionic properties, release of the core components from the particles can be time delayed as desired. Other methodologies also can be utilized to provide for time delayed release of the components at the core.

In some implementations, time delayed release of a core component can be measured from the time the particles are prepared, from the time of first delivery of the particles (e.g., the contact of the particle with an aqueous solution), or from the time that the particles first encounter the conditions of the desired delivery location (e.g., the conditions of a tagmentation reaction). Delayed release can be for a time of about 5 minutes or greater, 10 minutes or greater, 15 minutes or greater, 20 minutes or greater, 30 minutes or greater, or about 45 minutes or greater. In each instance, the maximum time of delayed release depends on the time it takes for the lysis reaction to occur.

In one or more implementations, the disclosure can relate the nature of the compositions and systems to the conditions to which they are subjected. More particularly, the compositions and systems can exhibit a first set of characteristics and/or functions under a first set of conditions and can exhibit a second set of characteristics and/or functions under a second set of conditions. The first set of conditions (which may be referred to as “standard conditions”) can be conditions under which the particles are prepared and/or stored, and the second set of conditions can include conditions present at the location where the particles are delivered. The first set of conditions, for example, can be approximately room temperature and pressure. The second set of conditions, for example, can be conditions encountered in a lysis reaction or a tagmentation reaction. As discussed above, release of reagents from the particles can be dependent upon the conditions encountered by the particles. Specifically, degradation of the particle may be substantially absent under the first set of conditions but be present under the second set of conditions. Similarly, diffusion may be substantially absent under the first set of conditions but be present under the second set of conditions. The second set of conditions may thus be characterized as the conditions under which microsphere release may proceed.

In some implementations, the conditions under which core release may proceed can particularly relate to temperature. For example, release may be provided at temperatures of about 50° C. or greater, about 60° C. or greater, about 70° C. or greater, or about 80° C. or greater. In some implementations, such temperatures can have an upper bound that is consistent with the average maximum temperature of a DNA library preparation reaction as described herein. In some implementations, release may be provided at temperatures of about 15° C. or less.

As further examples, the conditions under which release may proceed can particularly relate to pH. In particular, core release may proceed when the particles are subjected to a pH change (increase or decrease) of at least about 1, at least about 2, or at least about 4. The pH change can be a change of about 1 to about 12, about 1.5 to about 10, or about 2 to about 8.

The second set of conditions under which cargo release can occur can encompass any one of the conditions noted above in the ranges noted above. The second set of conditions under which cargo release can occur can encompass two or more of the conditions noted above in the ranges noted above. For example, core release can occur based on any one of the temperatures, pH ranges, and salt concentrations noted above. In some implementations, cargo release can occur when the particles are subject to any of the following combinations of conditions noted above, for example; temperature and pH; and temperature and salinity.

### Lyophilization

Methods known in the art can be utilized to lyophilize any materials, in particular, shelf-stable bioassay reagents. Typically, a pre-lyophilization formulation further contains an appropriate choice of excipients or other components such as stabilizers, buffering agents, bulking agents, and surfactants to inhibit or prevent a compound of interest from degrading (e.g., protein aggregation, deamidation, and/or oxidation) during freeze-drying and storage. The formulation for lyophilization can include one or more additional ingredients including lyoprotectants or stabilizing agents, buffers, bulking agents, isotonicity agents and surfactants.

After the substance of interest and any additional components are mixed together, the formulation is lyophilized. Lyophilization generally includes three main stages: freezing, primary drying and secondary drying. Freezing is necessary to convert water to ice or some amorphous formulation components to the crystalline form. Primary drying is the process step when ice is removed from the frozen product by direct sublimation at low pressure and temperature. Secondary drying is the process step when bounded water is removed from the product matrix utilizing the diffusion of residual water to the evaporation surface. Product temperature during secondary drying is normally higher than during primary drying.

Methods of lyophilization are described, for example, in Bjelošević, et al., (International Journal of Pharmaceutics, 576: 119029, 2020) herein incorporated by reference.

Assessment of lyophilization quality can be carried out by measuring glass transition temperature of maximally freeze-concentrated fraction (Tg′) and/or eutectic temperature. Glass transition temperature is the temperature at which an amorphous polymer changes from a hard/glassy state to a soft/leathery state, or vice versa. Tg is directly related to a material's strength and capabilities for a desired purpose. Generally, the product temperature should be several degrees below Tc and/or Tg′ to avoid collapse. Eutectic temperature is the lowest melting temperature a solution can achieve.

A rehydration (or reconstitution) solution, e.g., a sample with DNA, as used herein may include water, deionized water, saline solutions, acidic solutions, basic solutions, detergent solutions and/or buffers. In a preferred implementation, the rehydration solution is water or buffer. Additional additives as described herein may be provided in the rehydration solution to further improve control of release of microspheres.

In various implementations, a pH in the rehydration solution is between about 6.0 and about 10.0, or between about 7.0 and about 8.0. A pH of the rehydration solution may be, for example, about 6.0, about 6.5, about 7.0, about 7.5, about 8.0, about 8.5, about 9.0, about 9.5, about 10.0, or any amount therebetween. Rehydration time will vary depending on composition content and reaction conditions (e.g., reagents, temperature, pH). In various implementations, rehydration time may be between 0.1 seconds and 10 hours. For example, rehydration time may be about 0.1 seconds, 1 second, 10 seconds, 30 seconds, 45 seconds, 60 seconds, 5 minutes, 10 minutes, 12 minutes, 15 minutes, 20 minutes, 30 minutes, 40 minutes, 50 minutes, 60 minutes, 70 minutes, 80 minutes, 90 minutes, 2 hours, 5 hours, 8 hours, 10 hours, or any amount of time therebetween.

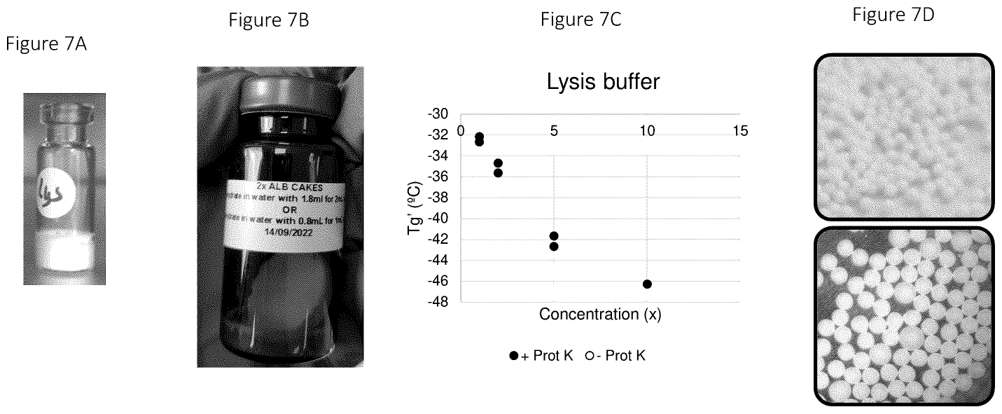

Lyophilizates can be analyzed for their physical appearance, and evaluated for their effectiveness in producing good quality libraries and other characterization studies after reconstitution. An exemplary visual cake appearance lysis and tagmentation lyophilizates of the present disclosure are provided in FIG. 7.

Lyophilization may be performed in a container, such as a tube, a bag, a bottle, a tray, a vial (e.g., a glass vial), syringe or any other suitable containers. The containers may be disposable. Lyophilization may also be performed in a large scale or small scale. In some instances, it may be desirable to lyophilize the protein formulation in the container in which reconstitution of the protein is to be carried out in order to avoid a transfer step. The container in this instance may, for example, be a 3, 4, 5, 10, 20, 50 or 100 cc vial.

As a general proposition, lyophilization will result in a lyophilized formulation in which the moisture content thereof is less than about 5%, less than about 4%, less than about 3%, less than about 2%, less than about 1%, and less than about 0.5%.

Also contemplated are means to stabilize the sample during collection of the sample and completion of the lysis and library preparation process. In various implementations, the sample, e.g., containing gDNA or other DNA, is stabilized with PEG6K, alkyl polyglucosides, salts, chaotropic agents (e.g. GITC, Urea), proteinase inhibitors, antioxidants (β-mercaptoethanol or dithiothreitol (DTT)), or buffers to stabilize pH. The stabilizer may be in the same container with the lyophilized cake or lyophilized microspheres, or in a different container or microsphere described herein.

In an alternative implementation, the sample is stabilized as a dried blood spot on a surface, which can be eluted in a container as described herein, e.g., a tube, multiwell plate, or other container.

### Smart Consumables

One aspect of the disclosure contemplates consumables for performing a molecular analysis workflow that are “smart” or have Automatic Identification and Data Capture (AIDC) capabilities (is “AIDC-capable”). In one implementation, the molecular analysis workflow is a next-generation sequencing workflow. As disclosed herein, an exemplary NGS workflow may comprise cell lysis & DNA extraction, optionally an isothermal PCR amplification step, library preparation/tagmentation, sequencing, imaging, and data analysis.

As used herein “smart” refers to an instrument, device, material or item, component, and/or part connected to other instruments, devices, materials or items, components and/or parts as part of a larger network and/or network cloud. Typically, a smart instrument, device, material or item, component, and/or part can be connected to other smart instruments, devices, materials or items, components, and/or parts through different wireless protocols or data transmissions such as, for example, Bluetooth, NFC, Wi-Fi, LiFi, 3G, etc., which can operate to some extent interactively and autonomously. As used herein, “network cloud,” “cloud,” or “the cloud” refers to a private, public or semi-public space that exists between the end points of a data transmission. In general, data that is transmitted enters the network cloud from one end point using a standard protocol and shares space in the network cloud with other data transmissions. Oftentimes, the data can also exit from the network cloud, where it may be encapsulated, translated and transported in myriad ways, in the same format as when it entered the network cloud.

In some implementations the AIDC method used in either or both the smart reaction substrate/holder and/or the smart reagent container is a smart label. In some implementations the AIDC method used in either or both the reaction substrate/holder and/or the reagent container is a Radio Frequency Identification (RFID) tag. Thus, in some implementations, the system includes a reaction substrate/holder with a smart label or RFID tag, and/or a reagent container with a reagent container smart label or RFID tag. In some implementations, when the system includes both a reaction substrate/holder, such as a reaction plate, with a reaction substrate/holder smart label or RFID tag, and one or more reagent container(s) with a reagent container smart label or RFID tag, the reaction substrate/holder smart label/RFID tag and the reagent container(s) smart label/RFID tag(s), together or collectively, can store and share information about the molecular analysis system. For example, system information can be stored or shared about samples, reagents, assays, users, and/or workflows used for an NGS workflow.

As used herein “RFID tag” refers to a part, such as a chip, that stores digital data and/or information. In some implementations, the tag comprises an integrated circuit and an antenna and a protective material that holds the pieces together and shields it from various environmental conditions. The protective material can depend on the application and RFID tags can come in a variety of shapes and sizes. The integrated circuitry may store data that can be communicated (e.g., sent or received) by a radio frequency transmitted by the antenna. The integrated circuit and antenna circuitry may be printed on the chip. An RFID tag can be read by an RFID reader using an antenna that emits radio frequencies to query the RFID tag. The term “RFID reader” as used herein includes RFID devices that can read information from and/or write information into an RFID tag. In some implementations, the RFID tag can be read only or read/write, and the information associated with the RFID tag can be hard-coded into the RFID tag at the time of manufacture or at some later time, or the RFID tag can contain information that is written to the RFID tag throughout its lifetime. In some implementations, the RFID tag is a “passive RFID tag” and does not have its own energy source, but responds to signals from a reader to transmit a signal. In other implementations, the RFID tag is an “active RFID tag” and comprises its own power source, such as a battery. A “writable RFID tag” is an RFID tag that has memory space that can be written to by an RFID writer. “Smart labels” are similar to RFID tags and can incorporate both RFID and barcode technologies. In some implementations, a smart label is made of an adhesive label embedded with an RFID tag, and may also include a barcode and/or other information. Some examples of RFID tags can be found in U.S. Pat. Nos. 6,147,662; 6,917,291; 5,949,049; 6,652,812; 6,112,152; and U.S. Patent Application No. 2003/0183683 all of which are herein incorporated by reference in their entireties for their disclosure of RFID tags, chips, labels, or devices, RFID readers, and RFID systems, their design and use.

In some implementations, the reaction substrate or reaction holder, as disclosed herein, includes, but is not limited, to a chamber, a channel, a card, an array, a vessel, a slide, or a plate. In various implementations, the reaction substrate or reaction holder may be a reaction substrate/holder with a plurality of reaction sites. Some examples of a reaction substrate or a reaction holder with a plurality of reaction sites may include, but are not limited to, a multi-well plate, such as a standard microtiter 96-well, a 384-well plate, or a microcard, or a substantially planar support, such as a slide, and open array, or an array. In some implementations, the reaction substrate or reaction holder may be made of glass or plastic or any other suitable material evident to those of skill in the art. The reaction sites, in various implementations of a reaction substrate or reaction holder, may include depressions, indentations, ridges, and combinations thereof, patterned in regular or irregular arrangements formed on the surface of the reaction substrate or a reaction holder.

In various implementations, the one or more reagent containers, as disclosed herein, may include, but is not limited to a vessel, bottle, tube, vial, well, or chamber or any combination thereof. In some implementations, the reagent container(s) may be made of glass or plastic or any other suitable material evident to those of skill in the art. The reagent container(s) may be of any size or dimension and may vary from one reagent container to the next, within the same system.

The smart consumables disclosed herein can include, for example, one or more smart reaction substrates or reaction holders (e.g., reaction plates or reaction arrays) and/or one or more smart reagent containers. Each of the smart reaction substrates or reaction holders can comprise a reaction substrate or a reaction holder RFID tag. Likewise, each of the reagent containers can comprise a reagent container RFID tag. Working together or separately, the reaction substrate or reaction holder RFID tag(s) and the reagent container RFID tag(s) can store and share various data or information. The reaction substrate or reaction holder RFID tag(s) and the reagent container RFID tag(s) can both send and/or receive information. An RFID tag reader can read information stored on an RFID tag. An RFID writer can write (or rewrite) information to an RFID tag.

The term “information” as used herein refers to data that can be stored electronically in the RFID tag and can be retrieved to be used as machine readable or human readable data for processing the biological reagent and/or carrier.

In some implementations, the smart consumable comprises pre-spotted reagent(s). “Pre-spotted,” as used herein, refers to reaction substrates or reaction holders comprising a reagent(s) that has been added to the reaction substrate or reaction holder (or pre-loaded) by a manufacturer of the reaction substrate or reaction holder and is not directly added to the reaction substrate or reaction holder by the user. Pre-spotted reaction substrates or reaction holders can also be considered to be ready-to-use. “Ready-to-use,” as used herein, can mean that only a limited number of additional reagents, or even no additional reagents, are needed to be added to the reagent(s) for a reaction to take place or can mean that only a liquid, such as water or a buffer, and/or a test sample may need to be added to the reagent(s) for a reaction to occur.

The RFID tag in the RFID tagged container can store data regarding; characteristics of a reagent in the container, identification of the reagent to be applied or released into the blood sample to start a reaction, the volume of the blood sample to be applied to the container, a date and/or time at which the blood sample is applied to the container, identification of the next reagent to be used in the workflow for the NGS workflow, and/or an interaction of a prespotted reagent(s) with the added blood sample in the workflow.

In one implementation, the smart consumable is an RFID tagged container pre-spotted with microspheres of a lysis reagent and core/shell particles of the tagmentation reagents of the present disclosure for an NGS workflow. In one implementation, the RFID tagged container are used for collecting a biological sample and carrying out DNA library preparation. In another implementation, the NGS workflow begins with the addition of a test sample by the user.

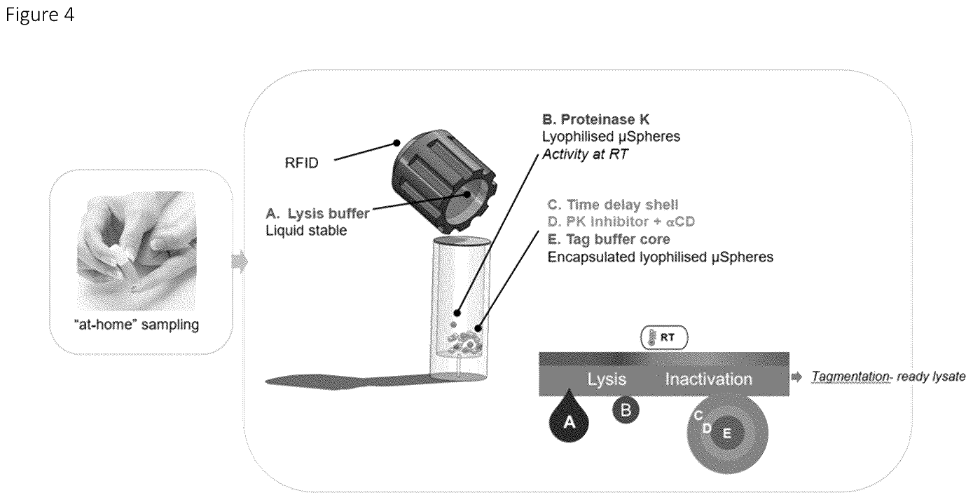

In one implementation, a container for collecting a sample comprises a plurality of lyophilized microspheres for carrying out a DNA library preparation reaction. The container may also comprise lysis buffer in the lid or cap of the smart consumable with RFID tag. The lyophilized microspheres may be encapsulated within a coated particle. In one implementation, uncoated lyophilized microspheres in the container comprise lysis reagents. In another implementation, coated particles comprising lyophilized microspheres in the container contain tagmentation reaction reagents. See FIG. 4.

In another implementation, a container for collecting a sample comprises a lyophilized cake for carrying out a DNA library preparation reaction. The container may also comprise lysis buffer in the lid or cap of the smart consumable with RFID tag. In one implementation, uncoated lyophilized microspheres in the container comprise lysis reagents. In another implementation, coated particles comprising lyophilized microspheres in the container contain tagmentation reaction reagents.

In another implementation, a container for collecting a sample comprises lyophilized beads, coated solid beads or stacked lyophilized cakes.

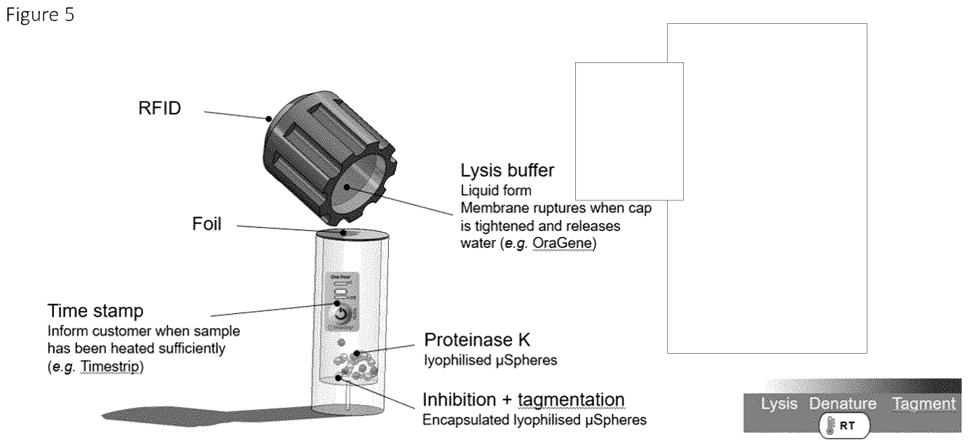

In an alternate implementation, the smart consumable can leverage transit time when shipping a sample to a sequencing lab to carry out the lysis and tagmentation reactions. In one implementation the container contains a time stamp or other indicator that notifies the consumer when the desired reactions e.g., tagmentation, gap-fill/extension-ligation, or first strand PCR synthesis, are completed. In one implementation, a container for collecting a sample comprises a plurality of lyophilized microspheres for carrying out a DNA library preparation reaction. The container may also comprise lysis buffer in the lid or cap of the smart consumable with RFID. The lyophilized microspheres may be encapsulated within a coated particle. In one implementation, uncoated lyophilized microspheres in the container comprise lysis reagents. In another implementation, coated particles comprising lyophilized microspheres in the container contain tagmentation reaction reagents. See FIG. 5.

In various implementations, the container or smart consumable is stored between about 4° C. and 30° C. at the inserting step, the sealing step and or the shipping step. In various implementations, the container or smart consumable is stored between about 4° C. and 8° C., between about 4° C. and 25° C., or between about 20° C. and 30° C. at the inserting step, the sealing step and or the shipping step.

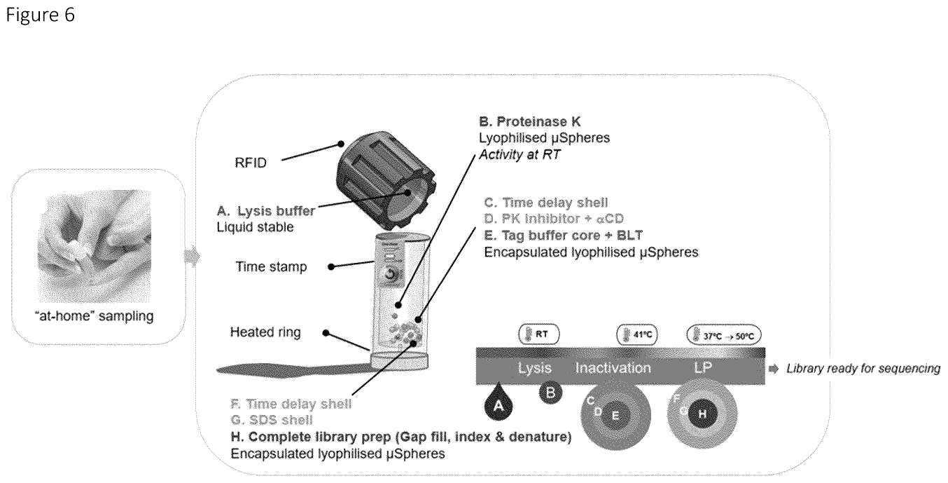

In another implementation, the smart consumable container comprises an apparatus that allows for full DNA library preparation. In one implementation the container comprises a heating element that allows for library preparation (gap fill, index and denaturing) in transit and a time stamp or other indicator that notifies the consumer when the desired reactions, e.g., tagmentation, gap-fill/extension-ligation, or first strand PCR synthesis, are completed. In one implementation, a container for collecting a sample comprises a plurality of lyophilized microspheres for carrying out a DNA library preparation reaction. The container may also comprise lysis buffer in the lid or cap of the smart consumable with RFID. The lyophilized microspheres may be encapsulated within a coated particle. In one implementation, uncoated lyophilized microspheres in the container comprise lysis reagents. In another implementation, coated particles comprising lyophilized microspheres in the container contain tagmentation reaction reagents. In a further implementation, the container comprises additional coated particles encapsulating lyophilized microspheres comprising. See FIG. 6.

In one implementation, the RFID tagged container may further comprise a heating element, a temperature sensor, a light sensor, and/or a motion sensor directly or indirectly coupled to the reagents, the container or the RFID tag, wherein the RFID tag stores a temperature history, a light exposure history, a motion detection history for the reaction substrate or reaction holder. In some implementations, the RFID tagged container further comprises a barcode. Exemplary RFID tagged containers are described in WO2006081612A1, US20170336428A1 & WO2009076011A1, incorporated herein by reference.

In one implementation, a heating mechanism provides at least one temperature zone with a predefined temperature either for the heat inactivation of the Proteinase K and/or as a thermal trigger-release mechanism for the core/shell particles of the disclosure. In a further implementation, the heating mechanism provides a temperature of about 65° C. Examples of suitable heating mechanisms include for example the use of sodium acetate technology, e.g., self-heating hot pots, that utilize a supersaturated solution of a metal salt, in particular an aqueous solution of sodium acetate (CH₃COONa), which releases exothermic heat upon crystallization of the solute into sodium acetate trihydrate (CH₃COONa·3H₂O) upon external mechanical stimulation such as strong shaking of the solution or sharp-tip impact applied for the initial nucleation of sodium acetate crystals.

In various implementations, the RFID tag is used to manage patient-specific information related to the sample throughout the entire process of collection, preparation, and analysis of said sample allowing sample identification and traceability. Optionally, the RFID tagged container further comprises a temperature, colorimetric or fluorescent indicator, which responds to an appropriate chemical species in the finished reaction by producing a observable change, thereby alerting the user that the reaction is complete. In one implementation, the colorimetric indicator could be incorporated in the encapsulated particle and gets released after digestion, wherein it is liberated with the enzyme as the trigger. In another implementation, the indicator could be incorporated into the shell such that it reacts with the blood lysate. In yet another implementation, the indicator could be immobilized on the walls of the RFID tagged container reacting either to the environmental condition changes, release of an intracellular component or from reaction from the release of a component from the encapsulated cores.

Data regarding characteristics of the reagents, blood sample and RFID tagged container can comprise one or more of the following: an ID number; an expiration date; a part number; a barcode; a lot number; a part type; a storage temperature and/or storage temperature range; a reagent concentration; a reagent form factor; a recommended reagent concentration and/or volume to use in the workflow; a provision for liquid transfer support; a sales order number; a reagent name; an assay name; an assay location for a reagent to be used on the reaction holder; an assay ID; a suggested or required protocol for the NGS workflow; a sample name; a master mix name; an internet link or address (url); a reaction and/or a reagent volume; a test sample name; an analysis setting for the molecular analysis; a sample type; a molecular analysis type; and an instrument run protocol. Data regarding characteristics of the reagents, blood sample and RFID tagged container can be written and/or rewritten to the reaction substrate or reaction holder RFID tag and/or the reagent container RFID tag. The reaction substrate or reaction holder RFID tag and/or the reagent container RFID tag may have a capacity to store at least 8 kilobytes of information.

In another implementation, the RFID container is tamperproof to minimize the risk of contamination that is widespread in alternative sampling systems for samples such as blood and semen. In one implementation, said tamper-proof container is sealed using incorporated tamperproof and tamper evident adhesive. Such adhesive may contain light or gas activated agents, which indicate tampering.

In various implementations, provided is a test to indicate that the lysis of the sample has progressed to completion. In various implementations, the test is a lateral flow test, a colorimetric test, or a temperature sensor. In certain implementations, the test is carried out inside the container. In other implementations, the test is carried out outside the container.

Provided herein are systems comprising the compositions as described herein. The system includes one or more compositions as described herein, and one or more containers, wherein the one or more compositions is placed in one or more containers under conditions effective to form a sequential system for preparing a DNA library. In various implementations, the system comprises one or more containers to hold the compositions, wherein the one or more containers include a PCR tube, vial, microtube, flow cell, multiwell plate, glass tube, transwell membrane/mesh insert, cartridge or microfluidic chip.

In various implementations, the composition, container, kits or methods comprise a lyophilized cake in combination with one or more lyophilized microspheres as described herein. In various embodiments, the lyophilized cake is in a multiwell plate, microtube, cartridge, or other container as described herein. In various implementations, the lyophilized cake is rehydrated with a rehydration solution. In various implementations, the rehydration solution is lysis buffer, and optionally a proteinase, as described herein, Methods of making lyophilized materials such as cakes or microspheres for use alone or in combination in a system or container for DNA sample processing are disclosed in US 2022/0331770, herein incorporated by reference.

The system may further include a temperature controller or sensor. The temperature controller may be used to change or adjust temperature of the system to further control release of various components of the compositions described herein. For example, the temperature controller may be used to speed up or slow down the release from the first or second shell. Similarly, the temperature controller may be used to speed up or slow down the release of the interior core to facilitate or control the release of one or more reagents. In one implementation, the system comprises a temperature controller on the container in the system. For example, the temperature controller may include a resistive heater proximate to a wall of the container, e.g., a cartridge, tube, chip or well, to provide heat thereto. The temperature controller may also include a temperature sensor. The temperature controller may also include circuitry to activate and deactivate the heater to maintain the well at a specified temperature.

### Kits

In another aspect, the present disclosure provides a kit comprising a container holding a lyophilized formulation, wherein the formulation can be reconstituted in about 15 minutes or less, and instructions for reconstituting the lyophilized mixture with a diluent to produce a reconstituted liquid formulation. In some implementations of this aspect of the invention, instructions are included in the kit for reconstituting the lyophilized formulation with a diluent. The kit can further comprise a second container which comprises a diluent.

The kit may comprise one or more receptacles (such as vials, ampoules, containers, tubes) of any appropriate shape, size and material (preferably waterproof, e.g. plastic or glass) containing the core/shell lyophilized compositions of the present invention in an appropriate dosage for DNA extraction and library prep. The kit may additionally contain directions for use (e.g. in the form of a leaflet or instruction manual), means for collecting a biological sample of the present disclosure such as a syringe.

### EXAMPLES

The following examples are intended to illustrate, and not intended to limit, the scope of the present disclosure as set forth in the appended claims.

### Example 1—Materials and General Methods

### Materials

Poly (lactic-glycolic) Acid (PLGA), DMSO, trehalose dehydrate, sucrose, maize starch, α-cyclodextrin, magnesium acetate, Tetrapeptidyl Chloromethyl Ketone (TCK, proteinase inhibitor, Cat #539470), Tris Acetate, Tween 20, EDTA, SDS, Tris HCl polyethylene glycol (PEG) 1500 and 4000 were purchased from Sigma (Sigma Chemical Company, St. Louis, MO). (Thermo Fisher Scientific, Waltham, MA, USA). The PEFABLOC® SC-Protease Inhibitor (Cat #11429868001) was purchased from MILLIPORE® (Millipore Corp., Bedford, MA). The thermolabile (TL) NEB proteinase K and non-thermolabile Proteinase K were purchased from New England Biolabs (Cat #s: P8111S & #P8107 respectively; NEB Ipswich, MA). Proteocut K was purchased from Biocatalysts (Cat #PK909L; Cardiff, Wales). Proteinase K and DNA-IQT paramagnetic particles were purchased from Promega, (Cat #s: V3021 and DC6701 respectively; Promega, Madison, WI). A Proteinase K was purchased from Sigma (Cat #. P4850; Sigma Aldrich St. Louis, MO). A Proteinase K was purchased from Roche (Cat #. 3 115 836; Roche Diagnostics GmbH). A Proteinase K was purchased from Zymo Research (Cat #. D30Jan. 2, 2020; Zymo Research Corp.). A protease was purchased from Qiagen (Cat. #:19157 QIAGEN GmbH, Hilden, Germany).

### Determination of Tg′ Values of Cryoprotectant Solutions

Glass transition temperature of maximally freeze-concentrated fraction (Tg′) for cryoprotectants at 10% w/v was determined in 0.1% PVA aqueous solutions with differential scanning calorimetry (DSC). The measurements were performed on a DSC4000 Perkin-Elmer calorimeter, under a nitrogen atmosphere, using the cycle of cooling the solution from +20 to −70° C. at 2° C./min, and subsequently a reheating cycle back to +20° C. at 2° C./min. The instrument was calibrated for temperature and heat flow with two point calibration method using indium and zinc reference samples and scan rate 10° C./min prior to measurements of studied samples.

### Example 2—Compatibility Assessment of Various Proteinase Ks Used in DNA Library Preparation

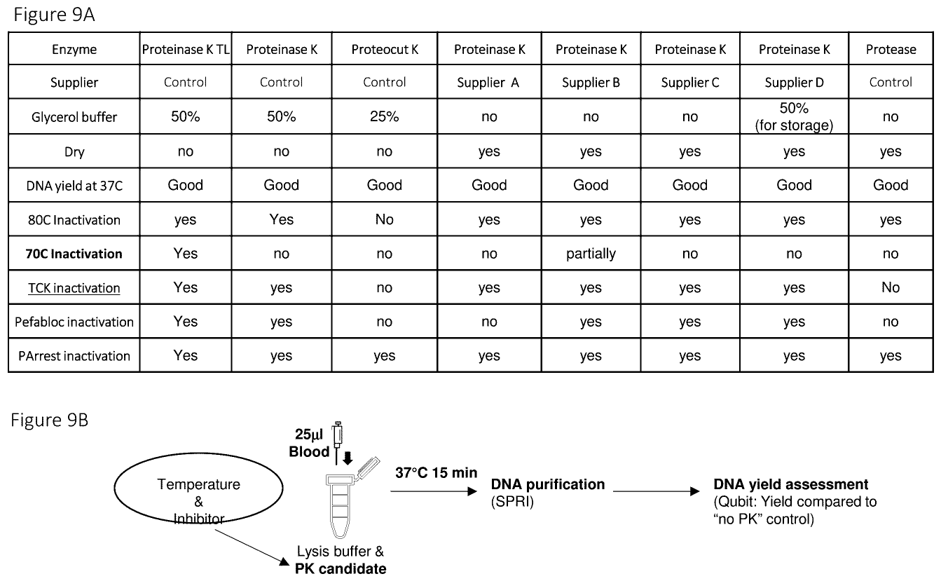

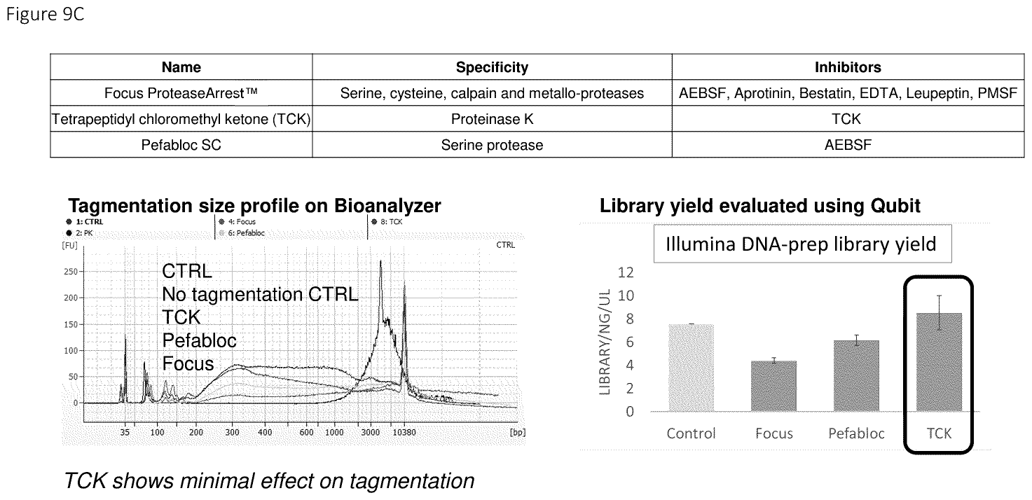

In order to produce high quality lyophilized microspheres for sample preparation, it is beneficial for a reagent to function exceptionally well in several respects. The reagent should effectively isolate a pure DNA sample from a variety of sample types and result in the highest possible yields of DNA. It should be lyophilization-compatible (lyo-compatible), e.g., glycerol-free. It should be compatible both with the other components in the DNA extraction step and thermolabile for easy inactivation before the tagmentation step (e.g., at 70-80° C.) or efficiently inactivated by a protease inhibitor. It should be user friendly, meaning the steps must not be too onerous, and the components should not be toxic and can be disposed of easily. FIG. 9A shows the characteristics of the various proteinase K enzymes evaluated herein.

The proteases from four suppliers, Suppliers A-D, do not contain glycerol buffer and are thus lyo-compatible (FIG. 9A). Moreover, they are all inhibited by the PK inhibitor (TCK), which showed the least interference on the tagmentation reaction (FIG. 9C).

### Example 3—Sigma PK Showed the Best Results in the “One-Pot” Lyophilized Assay of the Present Disclosure

A blood sample was taken from a human subject. Cells were lysed to allow DNA release from 25 μL of the sample using the “one-pot” core/shell reagents comprising the Proteinase K from NEB, and the other 4 Proteinase Ks that had been found to be most compatible with the “one-pot” assay (i.e., Suppliers A-D). The blood lysate was loaded onto a custom-made NovaSeq S1 flow cell (ILLUMINA®, San Diego, CA, USA) and the library prep performed on said flow cell according to manufacturer's instructions.

Insert size: Based on the alignments of the reads to the reference genome, the insert sizes of the different libraries was calculated. The average insert sizes of the libraries prepared using TL NEB, Supplier C-D ranged between 362-381 bp. The Supplier A-B libraries had average insert sizes of 430 and 446 bp respectively. All candidate PKs generated insert sizes that are well above 300 bp, which allows an optimal use of 151×151 paired-end reads.

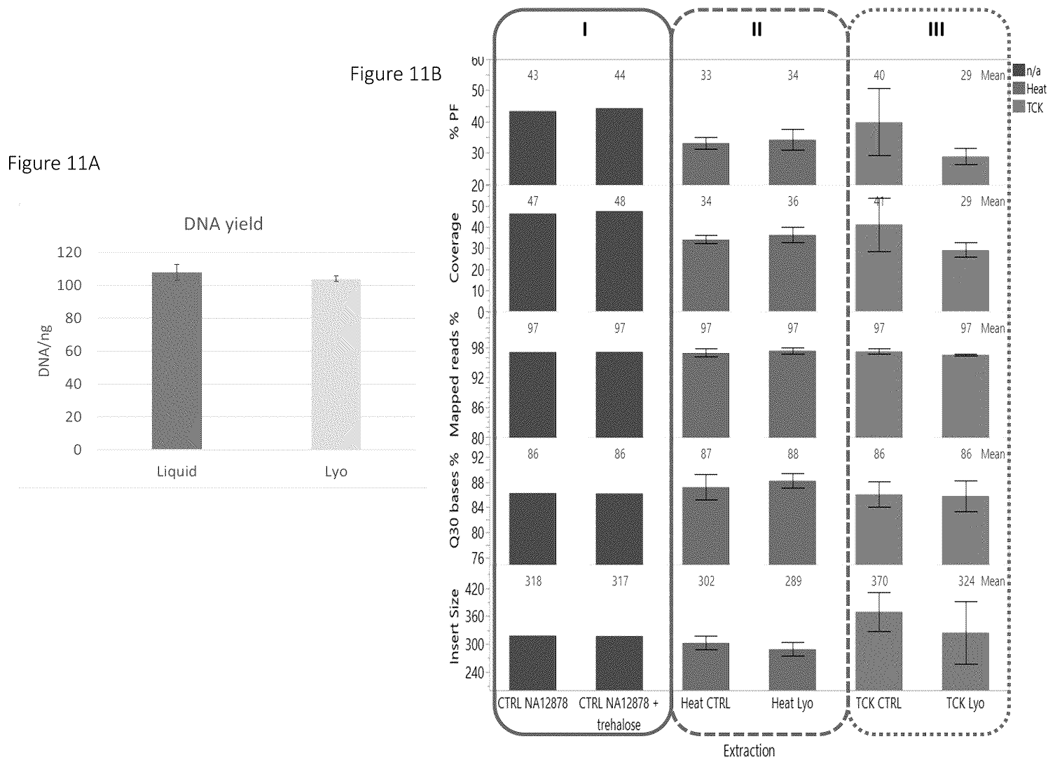

% Q30: Base calling accuracy, measured by the Phred-like quality score (Q score), is a common metric used to assess the accuracy of a sequencing run. A higher quality score indicates a lower probability that an individual base is called incorrectly. Currently, the Q30 score, which represents a 1/1000 chance of an incorrect base identification (Ewing, B., & Green, P., 1998, Genome research, 8(3): 186-194; Ewing et al., 1998 Genome Res, 8(3): 175-85), is the de facto standard for measuring the accuracy of NGS reads. Q30 is equivalent to the probability of an incorrect base call 1 in 1000 times. This means that the base call accuracy (i.e., the probability of a correct base call) is 99.9%. A lower base call accuracy of 99% (Q20) will have an incorrect base call probability of 1 in 100, meaning that every 100 base pair sequencing reads will likely contain an error. When sequencing quality reaches Q30, virtually all of the reads will be perfect, having zero errors and ambiguities. All five libraries in this experiment passed this criterion, the lowest value was from the Supplier A PK library (77%), while the other four libraries had comparable %≥Q30 scores ranging from 85%-89% (FIG. 11).

Coverage: Coverage depth refers to the average number of sequencing reads that align to or “cover” each base in the sequenced sample. The Lander/Waterman equation is a method for calculating coverage (C) based on your read length (L), number of reads (N), and haploid genome length (G): C=LN/G. The libraries generated with TL NEB and Supplier B PKs had high and comparable genome coverage of 48×. The libraries generated with Supplied A, C and D PKs had lower genome coverage of 34×, 23×, and 15× respectively.

Mapped reads %: Mapped read percentage refers to the percentage of reads that are aligned to the reference genome. All the libraries had >96% of total reads mapped to the reference genome, though the library generated with the Supplier A protease showed a slightly decreased mapping rate of 86% that also had the most variability. For a very good library, the mapped reads % exceeds 90%, and for good libraries it should be above 80%.

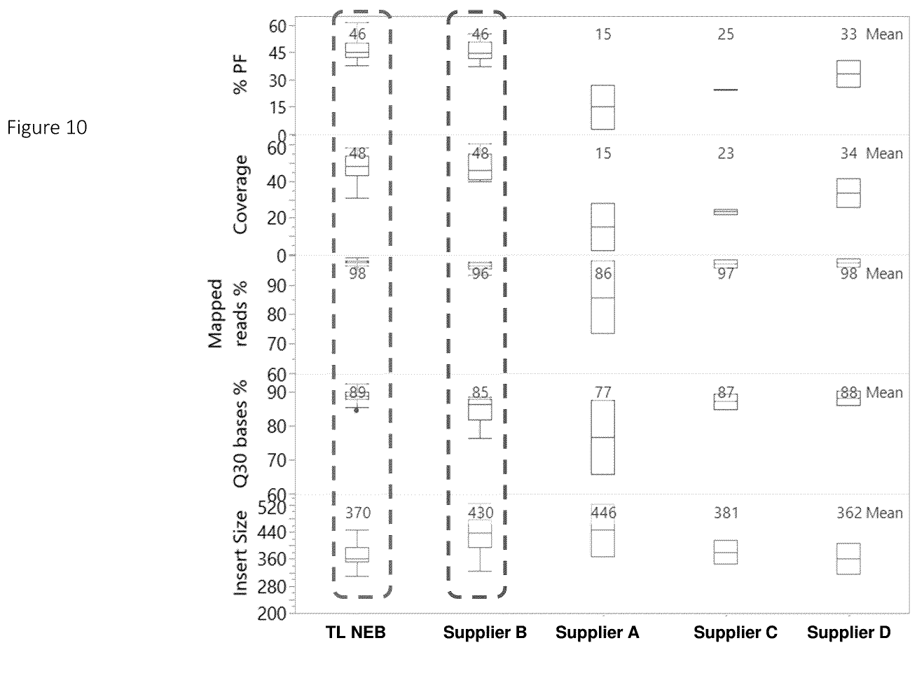

Passing filter (% PF): Passing filter (PF) is the metric used to describe clusters which pass a chastity threshold and are used for further processing and analysis of sequencing data. The % PF calculation involves the application of a chastity filter to each cluster. “Chastity” is defined as the ratio of the brightest base intensity divided by the sum of the brightest and second brightest base intensities. Clusters “pass filter” if no more than 1 base call has a chastity value below 0.6 in the first 25 cycles. This filtration process removes the least reliable clusters from the image analysis results. As such, a higher % passing filter (% PF) result indicates an increased yield of usable sequencing data. In the present study, the Supplier B PK library showed consistently good results with a comparable % PF to the TL NEB PK control library of 46% (FIG. 10). The Supplier D PK runs (n=2) had a % PF of 33% indicating that this PK might be another option although more runs may be beneficial for a more conclusive evaluation (FIG. 9). The Supplier A and D PK libraries had low % PF values (15%, and 25% respectively) pointing to possible issues with the library prep (FIG. 9).

Overall, this study identified Supplier B PK as the best performing PK relative to the control TL NEB (FIG. 10).

### Example 4—Effect of Coating on Lyophilization and Trigger Coatings

A benefit of the present method is that multiple lyophilized microspheres or particle encapsulating lyophilized microspheres can be within a sample collecting container in order to reduce the steps used to complete the DNA library preparation workflow.

In certain implementations, coating polymers can be incorporated that provide a desired time release of lyophilized microspheres from a particle. For example, for a 2 minute time delayed release (time delay linked to shell thickness) the components can be HPMC, Hydroxy ethyl/propyl cellulose, polyethylene glycol, PVP-co-PVAc, Eudragits, Isoleucine, Eudragit RL/RS, or Opadry CA, polyester (i.e., PLGA). For a 15 minute time trigger release, the material can be methyl/ethyl cellulose, cellulose acetate (CA) and/or PLGA. Other release triggers can be temperature triggered, e.g., with a Prot K inactivation at 55° C. with a potential material of wax, fatty acid, fatty acid ester, or upper critical soluble temperature (USCT) polymer, such as poly(acrylamide-co-acrylonitrile), poly(N-acryloyl glycinamide), and poly(N-isopropyl acrylamide) co-polymer.

It is contemplated that during the lysis phase of the reaction, there is no release trigger. However, to initiate the tagmentation reaction, a release trigger of 15 minute time delay, or a temperature release trigger are employed to release the tagmentation lyophilized microspheres into the reaction container.

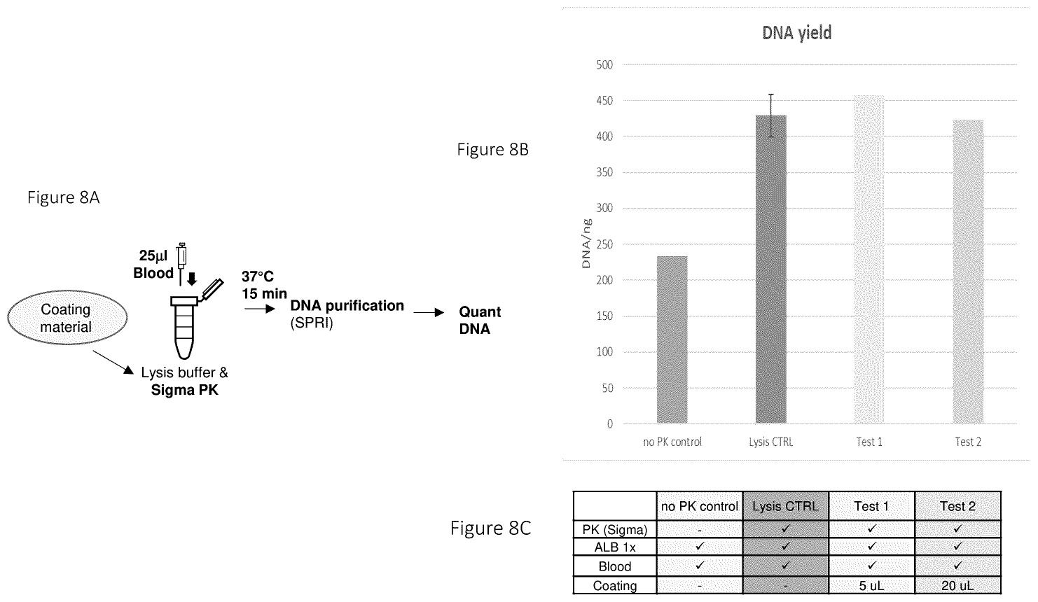

Next, it was hypothesized that it may be possible to have enzymes coated on the surface of the microspheres and is the coating material used for microspheres compatible with PK. Particle Coating material was made using VA64, Protect, Efka, Makon, ethanol, H2O)+PK. The coatings were tested on a functional assay, i.e., standard extraction, where PK activity can be assessed. FIG. 8 shows that DNA yield is similar to control when spiking in 5 and 20 μL of coating material.

### Example 5-Lyophilization and the Lyophilization Formulation do not Reduce the Activity of the Sigma PK

The effect of lyophilization and the composition of the lyophilization formulation using Sigma's PK enzyme were tested by comparing DNA yields from extractions comprising a liquid formulation or the lyophilized Sigma PK immediately after lyophilization/reconstitution. Both formulations extracted DNA essentially equally well as there was no statistical difference in the DNA extraction yields performed using the liquid or lyophilized Sigma PK formulation (FIG. 11A). Moreover, libraries prepared from DNA extracted using the lyophilized Supplier B PK lysis buffer showed equivalent sequencing performance compared to libraries prepared from DNA extracted using the liquid Supplier B PK formulation (FIG. 11B). The effect of trehalose (used in the lyophilization formulation) was tested by spiking this reagent on a control sample (NA12878) which was extracted using standard methods (e.g. Qiagen QIAmp DNA blood Mini Kit) and it had no significant impact on the sequencing performance of this library (FIG. 11B I). PK heat inactivation was also tested by sequencing libraries prepared using the control liquid formulation and the reconstituted lyophilized Supplier B PK formulation and PK heat inactivation. The use of the different formulations had no significant impact on the sequencing performance of the two libraries (FIG. 11B II) when heat is used for inactivation. However, the presence of trehalose seems to interfere with TCK inactivation of the Sigma PK (FIG. 11B III). It is hypothesized that different reagent combination, e.g., combinations of trehalose with new excipients (e.g., PVP 40K, Dextran 40K), may resolve this problem.

### Example 6-Assessment of the Compatibility of the Tagmentation and Lyophilization Reagents by Testing their Impact on Lyophilization and Sequencing Metrics

Cyclodextrins form complexes with hydrophobic and amphiphilic molecules, such as detergents. α-Cyclodextrin is often used in tagmentation buffer in combination with proteinase K inhibitors, such as TCK, where it chelates the SDS from the lysis buffer. However, the TCK inhibitor is more soluble in organic solvents, e.g., 100% DMSO. Unlike aqueous samples however, organic solvents are difficult to freeze and benefit from dilution prior to freezing. Moreover, owing to DMSO's physical properties, there are conflicting reports on its effect on lyophilization. On one hand, DMSO's high boiling point (189° C.) and low vapor pressure at room temperature (0.08 kPa at 25° C.), make it unsuitable for evaporation, however, its high freezing point (18.4° C.) means it can be effectively sublimated (Jakubowska et al., 2022. J Drug Deliv Sci Technol, 74:103528). In addition, DMSO is a polar organic compound and depresses the freezing temperature of water by distorting its hydrogen bonding, thereby inhibiting ice formation. As a result, lyophilization of DMSO/water mixtures may be particularly challenging depending on the composition, since the eutectic point for this organic solvent at a content between 50 and 70% is approximately −70 to −60° C. (Jakubowska et al., 2022. J Drug Deliv Sci Technol, 74:103528).

TABLE 4A
Tagmentation buffer formulations with different DMSO
concentrations for differential scanning colorimetry (DSC) study

| | | Conc. in | Conc. in |
|---|---|---|---|
| | Components | 1X buffer | 2X buffer |
| Buffer 1 | DMSO | 0% | 0% |
| | Tris Acetate | 10 mM | |
| | Magnesium Acetate | 5 mM | |
| | α-Cyclodextrin | 2.7% | |
| Buffer 2 | DMSO | 0.5% | 0.1% |
| | Tris Acetate | 10 mM | |
| | Magnesium Acetate | 5 mM | |
| | α-Cyclodextrin | 2.7% | |
| Buffer 3 | DMSO | 1% | 2% |
| | Tris Acetate | 10 mM | |
| | Magnesium Acetate | 5 mM | |
| | α-Cyclodextrin | 2.7% | |
| Buffer 4 | DMSO | 1% | 2% |
| | Tris Acetate | 10 mM | |
| | Magnesium Acetate | 5 mM | |
| | α-Cyclodextrin | 2.7% | |
| Buffer 5 | DMSO | 2.5% | 5% |
| | Tris Acetate | 10 mM | |
| | Magnesium Acetate | 5 mM | |
| | α-Cyclodextrin | 2.7% | |

TABLE 4B
Tagmentation buffer formulations with different
TCK & DMSO concentrations

| | | Conc. in | Conc. in |
|---|---|---|---|
| | Components | 1X buffer | 2X buffer |
| Buffer | DMSO & | 1.7% & | 3.4% & |
| 1 | TCK | 0.08 mg/mL | 0.16 mg/ml |
| | Tris Acetate | 10 mM | 20 mM |
| | Magnesium | 5 mM | 10 mM |
| | Acetate | | |
| | α-Cyclodextrin | 2.7% | 5.4% |
| Buffer | DMSO & | 0.25% & | 0.5% & |
| 2 | TCK | 0.08 mg/mL | 0.16 mg/mL |
| | Tris Acetate | 10 mM | 20 mM |
| | Magnesium | 5 mM | 10 mM |
| | Acetate | | |
| | α-Cyclodextrin | 2.7% | 5.4% |
| Buffer | DMSO & | 0.2% & | 0.4% & |
| 3 | TCK | 0.07 mg/mL | 0.14 mg/mL |
| | Tris Acetate | 10 mM | 20 mM |
| | Magnesium | 5 mM | 10 mM |
| | Acetate | | |
| | α-Cyclodextrin | 2.7% | 5.4% |
| Buffer | DMSO | 0.08% & | 0.16% & |
| 4 | | 0.08 mg/mL | 0.16 mg/mL |
| | Tris Acetate | 10 mM | 20 mM |
| | Magnesium | 5 mM | 10 mM |
| | Acetate | | |
| | α-Cyclodextrin | 2.7% | 5.4% |

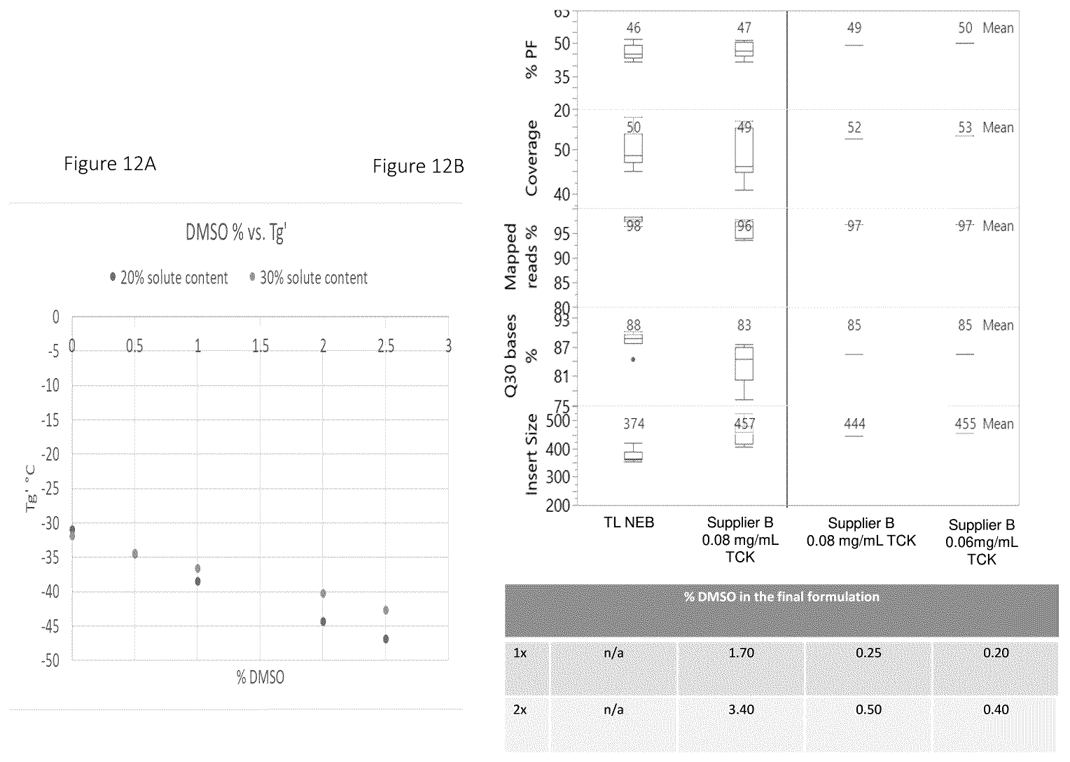

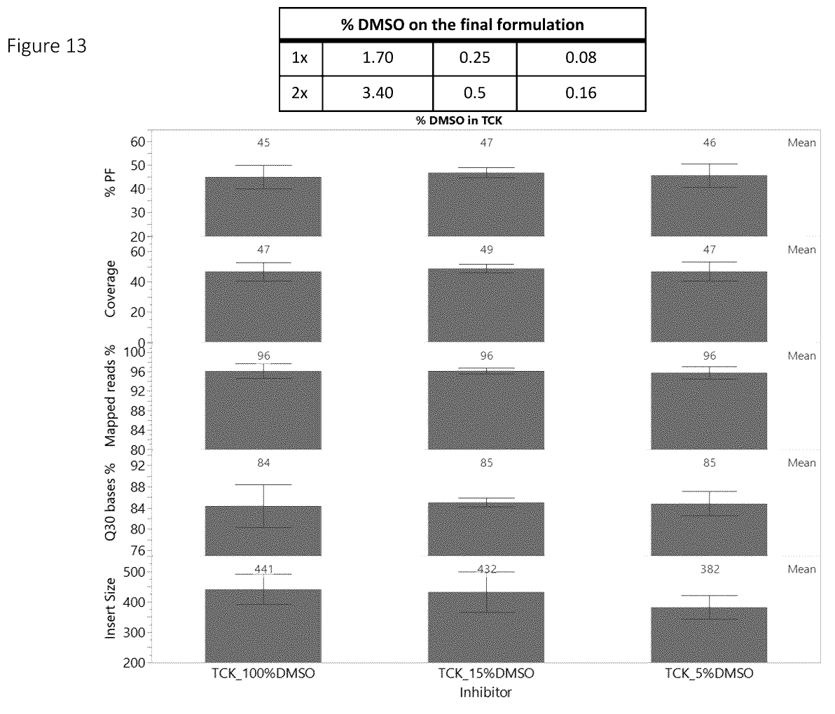

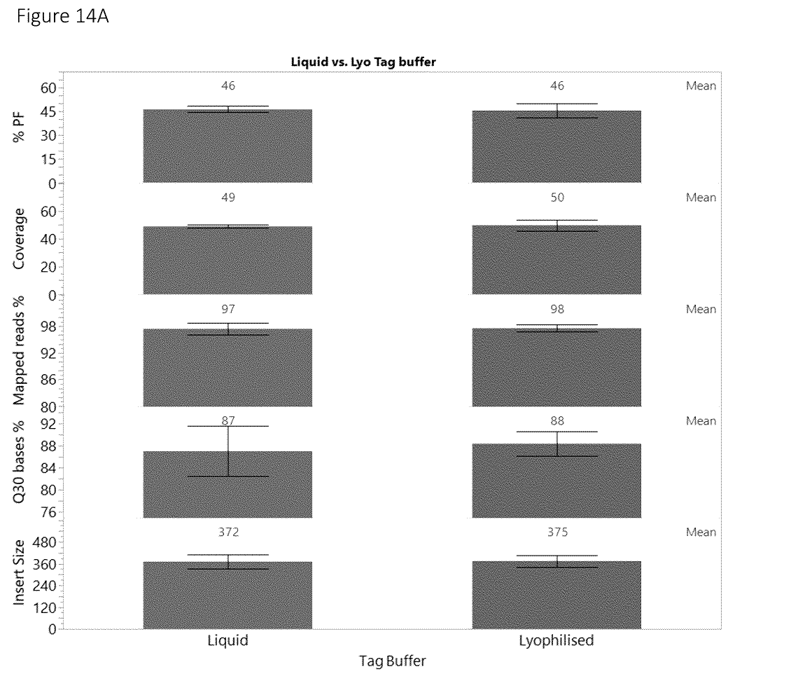

To assess the effect of DMSO on the lyophilization of the tagmentation buffers, and to determine the best concentration of DMSO to use, five tagmentation buffers comprising different DMSO concentrations were formulated (Buffer 1-5, Table 4A), and their Tg′ measured by differential scanning calorimetry (DSC). A Tg′ that is >−40° C. is desirable since this is a common primary drying temperature in typical lyophilization processes. This study showed a DMSO concentration of 1% gave a Tg′ that is >−40° C., making it the most suitable concentration for the formulation. Sequencing metrics from three test libraries prepared using the Sigma Proteinase K and different concentrations of the TCK, and DMSO concentrations; 0.08 mg/mL TCK & 1.7% DMSO, 0.08 mg/mL TCK & 0.25% DMSO, and 0.07 mg/mL TCK & 0.2% DMSO, were comparable to the metrics of the control run, which was prepared using the TL NEB PK (Table 4B, FIG. 12B). This means the % DMSO on the final 2× formulation can be reduced to 0.4% which would allow sample buffer lyophilization. Since 0.7% and 0.2% DMSO concentrations perform the same, this indicated that the DMSO concentration could be reduced further. Buffer 4 (Table 4B) is an exemplary tagmentation buffer formulation with a lower DMSO concentration. Sequence runs of libraries prepared from buffers containing TCK:0.7% DMSO, TCK: 0.25% DMSO, and TCK:0.08% DMSO all had comparable quality metrics (FIG. 13). The differences in insert sizes observed here was attributable to the batch of TSM used, and not the TCK.

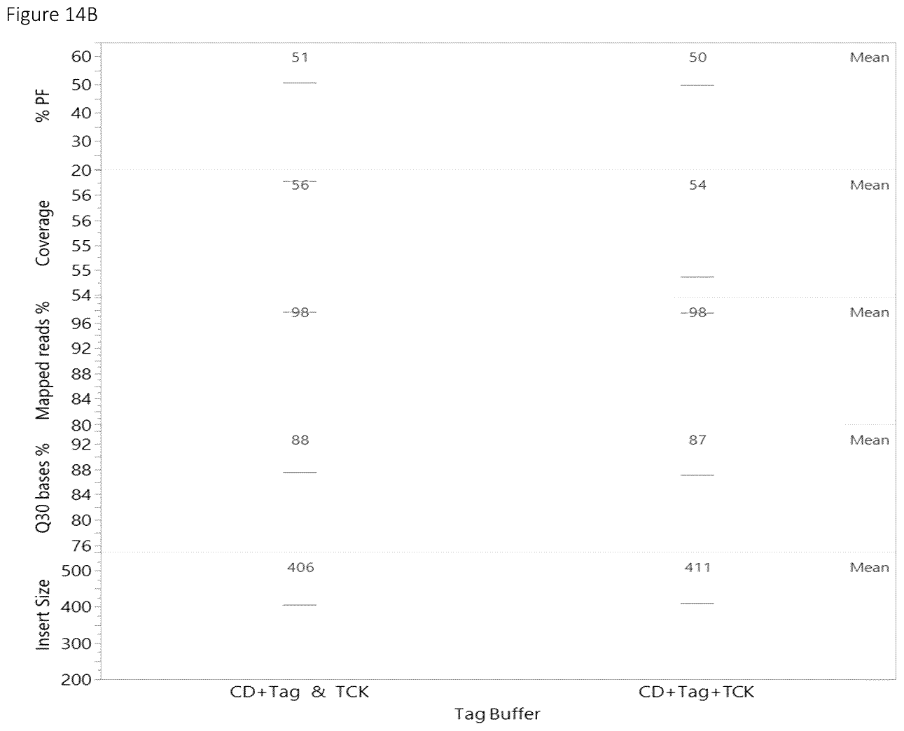

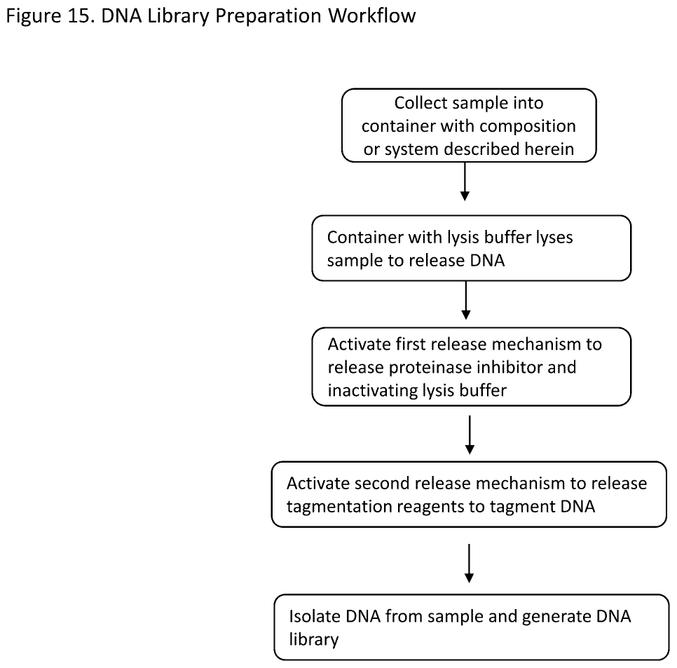

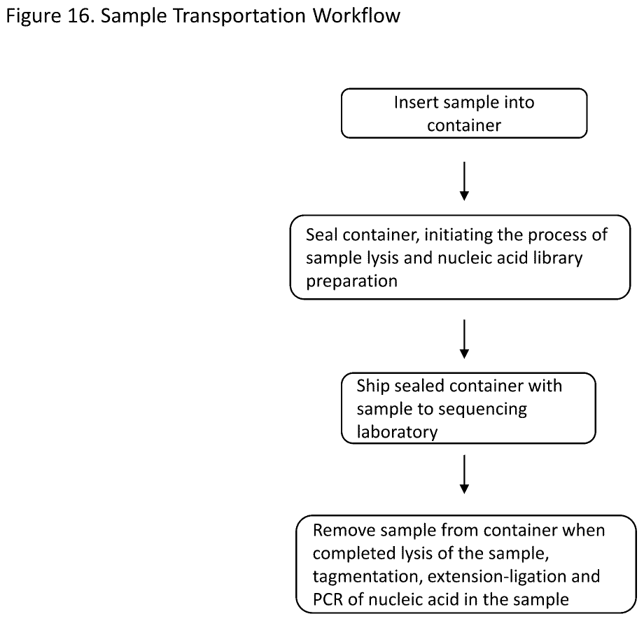

Cyclodextrin and TCK can be included in the tagmentation buffer: To assess the compatibility of the sequencing performance of the tagmentation buffer, the sequencing metrics of libraries prepared with lyophilized vs liquid tagmentation buffer (without cyclodextrin) were compared. The lyophilization of the buffer did not significantly impact the sequencing metrics and is therefore compatible with the formulation (FIG. 14A). To assess the compatibility of TCK on the sequencing performance of the tagmentation buffer, the sequencing metrics of libraries prepared with 3 components (CD, Tag buffer components & TCK) was assessed. FIG. 14B shows that the reagents appear to be compatible when lyophilized together.

### Example 7-Effects of Proteinase K on De-Escalating Blood Samples

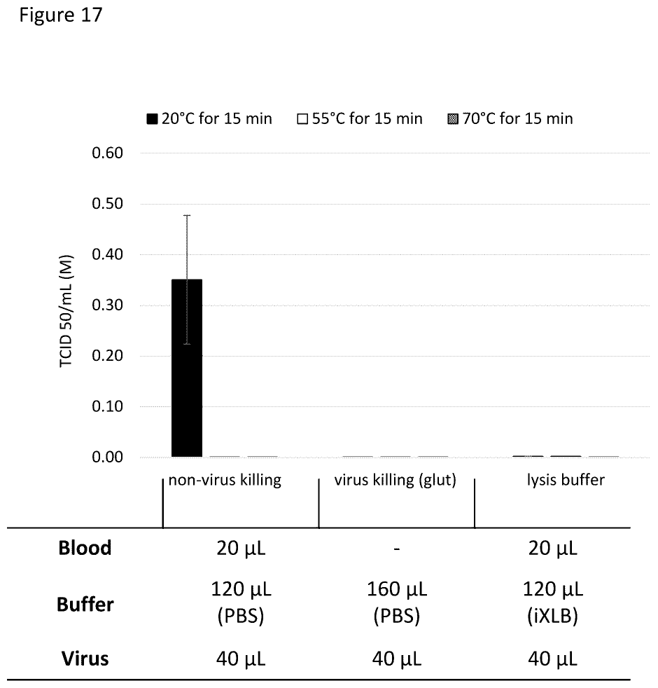

Next it was determined if the lysis buffer containing PK could inactivate virus that can contaminate blood samples. 25 μL blood containing Vaccinia virus was collected into 225 μL lysis buffer containing PK and incubated for 15 minutes at 20° C., 55° C. or 70° C. After 5 days, levels of virus in the samples were determined. Results show that Vaccinia virus in blood was inactivated at >99% in lysis buffer alone and fully deactivated in lysis buffer at >55° C. for 15 minutes. (FIG. 17).

The present invention has been described by way of illustration and with reference to specific examples and implementations. However, this application is intended to cover those changes and substitutions which are apparent and may be made by those skilled in the art without departing from the spirit and scope of the claims. All of the references cited herein are incorporated by reference.

1. A system comprising a container with an opening to receive a biological sample, wherein the container comprises a workflow reagent release system, comprising:
a lyophilized material comprising lysis buffer and a proteinase; and
a plurality of first particles comprising
a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; and,
a second outer shell which encapsulates a second inner core comprising one or more lyophilized microspheres comprising one or more reagents for tagmentation of DNA, wherein the second outer shell releases the second inner core in response to a second release trigger mechanism.

2-4. (canceled)

5. The system of claim 1, wherein the first particle comprises a third outer shell which encapsulates a third inner core, wherein the third inner core comprises workflow reagents for extension-ligation and PCR, wherein the third outer shell releases the third inner core in response to a third release trigger mechanism; or
further comprising a plurality of second particles comprising a third outer shell which encapsulates a third inner core, wherein the third inner core comprises workflow reagents for extension-ligation and PCR, wherein the third outer shell releases the third inner core in response to a third release trigger mechanism.

6-7. (canceled)

8. The system of claim 1, wherein the first trigger release mechanism is the biological sample dissolving the lyophilized microspheres, thereby forming a lysis solution.

9. The system of claim 1, wherein the lyophilized material comprises one or more reagents for lysing cells contained in the biological sample, optionally wherein the one or more reagents for lysing cells is selected from the group consisting of a phosphate buffer solution, a salt, a detergent, an alcohol, a protease, a lysis buffer, a lyoprotectant, or a combination thereof.

10-11. (canceled)

12. The system of claim 9, wherein the proteinase is a broad-spectrum serine proteinase.

13-14. (canceled)

15. The system of claim 1, wherein the first or second release trigger is a temperature-controlled release mechanism, a pH-controlled release mechanism, a time-controlled release mechanism, a position-controlled release mechanism, or any combination thereof.

16. (canceled)

17. The system of claim 1, wherein the second inner core comprises lyophilized microspheres comprising one or more tagmentation reagents, optionally wherein the one or more tagmentation reagents are selected from the group consisting of a Tn5 transposase enzyme, one or more transposons, linker sequences, Tn5 2× Tagmentation Buffer, Mg2+, an SDS chelating agent, primers with transposome, a lyoprotectant and optionally, a Proteinase K inhibitor.

18. (canceled)

19. The system of claim 17, wherein the SDS chelating agent is a cyclodextrin (CD) selected from the group consisting of α-CD, β-CD, and γ-CD.

20. (canceled)

21. The system of claim 9, the lyoprotectant is selected from the group consisting of mannitol, sorbitol, inositol, sucrose, glucose, mannose and trehalose.

22. (canceled)

23. The system of claim 1, wherein the lyophilized material is a plurality of microspheres.

24. (canceled)

25. A composition comprising:
a plurality of first lyophilized microspheres comprising lysis buffer and a proteinase; and
a plurality of first particles comprising
a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; and,
a second outer shell which encapsulates a second inner core comprising one or more lyophilized microspheres comprising one or more reagents for tagmentation of DNA, wherein the second outer shell releases the second inner core in response to a second release trigger mechanism.

26-45. (canceled)

46. A method comprising;
A. collecting a biological sample from a subject and placing the sample into a container with an opening, wherein the container comprises a workflow reagent release system;
wherein the workflow reagent release system comprises
a. lyophilized material comprising lysis buffer and a proteinase; and
b. first particles comprising
a first outer shell which encapsulates a first inner core, wherein the first inner core comprises one or more lyophilized microspheres comprising an inhibitor of the proteinase and a detergent chelator, wherein the first outer shell releases the first inner core in response to a first release trigger mechanism; and
a second outer shell which encapsulates a second inner core comprising one or more lyophilized microspheres comprising one or more reagents for tagmentation of DNA, wherein the second outer shell releases the second inner core in response to a second release trigger mechanism;
wherein the biological sample interacts with the lyophilized lysis buffer in the container resulting in release of nucleic acid from cells in the biological sample, and
allowing the lysis buffer to react for a period of time sufficient to carry out lysis of cells in the biological sample;
B. inactivating the lysis reaction (A) after the period of time by activating the first release trigger mechanism to release the proteinase inhibitor and detergent chelator from the first inner core, and allowing the inactivation reaction to proceed for a period of time sufficient to inactive the proteinase and to chelate the detergent; and
C. stopping the inactivation reaction (B) by activating the second release trigger mechanism to release the reagents for tagmentation of DNA from the second inner core, and allowing the tagmentation reaction to proceed for a period of time sufficient to tag the nucleic acid from the biological sample.

47-67. (canceled)

68. The method of claim 46, wherein contacting the sample with the lyophilized material comprising a lysis reagent generates a cell lysate, wherein the lysis reagent has one or more proteases, and wherein the cell lysate contains a target nucleic acid.

69. The method of claim 46, wherein the release of the tagmentation reagents applies at least one transposase and at least one transposon end composition containing a transferred strand under conditions where the target nucleic acid and the transposon end composition undergo a transposition reaction to generate a mixture,
wherein,
the target nucleic acid is fragmented to generate a plurality of target nucleic acid fragments, and
the transferred strand of the transposon end composition is joined to 5′ ends of each of a plurality of the target nucleic acid fragments to generate a plurality of 5′ tagged target nucleic acid fragments.

70. (canceled)

71. A container for collecting a biological sample, the container comprising a workflow reagent release system of claim 1, wherein the container comprises an indicator that changes upon completion of the workflow reagent release system inside the container.

72-78. (canceled)

79. A method of transporting a sample for preparation of a nucleic acid library, comprising;
inserting the sample into a container comprising a system of claim 1;
sealing the container and allowing the system to begin the process such of sample lysis and nucleic acid library preparation;
shipping the sealed container to a nucleic acid sequencing laboratory such that upon arrival at the sequencing laboratory, the system has completed lysis of the sample, tagmentation of nucleic acid in the sample, extension-ligation and PCR of nucleic acid in the sample.

80-87. (canceled)

88. A method comprising;
placing a sample into a container, wherein the container comprises a workflow reagent release system, wherein the workflow reagent release system comprises
lyophilized material comprising lysis buffer and a proteinase;
a first particle having a first outer shell which encapsulates a first inner core, wherein the first inner core comprises one or more lyophilized microspheres comprising a proteinase inhibitor and a detergent chelator, wherein the first outer shell releases the first inner core in response to a first release trigger mechanism; and
a second particle having a second outer shell which encapsulates a second inner core comprising one or more lyophilized microspheres comprising one or more reagents for tagmentation of DNA, wherein the second outer shell releases the second inner core in response to a second release trigger mechanism;
wherein the sample interacts with the lysis buffer in the container resulting in release of nucleic acid from cells in the biological sample;
releasing the proteinase inhibitor and the detergent chelator from the first inner core by activating the first release trigger mechanism; and
releasing the one or more reagents for tagmentation of DNA from the second inner core by activating the second release trigger mechanism.

89. (canceled)

90. The method of claim 88, further comprising isolating the nucleic acid and generating a nucleic acid library using a library preparation kit.

91-110. (canceled)

111. The method of claim 88, wherein releasing the one or more reagents for tagmentation of DNA applies at least one transposase and at least one transposon end composition containing a transferred strand under conditions where a target nucleic acid and the transposon end composition undergo a transposition reaction to generate a mixture,
wherein,
the target nucleic acid is fragmented to generate a plurality of target nucleic acid fragments, and
the transferred strand of the transposon end composition is joined to 5′ ends of each of a plurality of the target nucleic acid fragments to generate a plurality of 5′ tagged target nucleic acid fragments.

112. (canceled)

113. The system of claim 1, wherein the lyophilized material is a lyophilized cake.
